(19) United States
(12) Patent Application Publication
(10) Pub. No.: US 2007/0225202 A1
(43) Pub. Date: Sep. 27, 2007

(54) Methods for Identifying Modulators of Active Kit Tyrosine Kinase Receptor

(75) Inventors: Julian Andreev, Weymouth, MA (US); Brian Healey, Scituate, MA (US); Peter Blume-Jensen, Newton, MA (US); Stephen J. Arkinstall, Belmont, MA (US); Rong Dong, Wellesley, MA (US)

Correspondence Address: MARSHALL, GERSTEIN & BORUN LLP, 233 S. WACKER DRIVE, SUITE 6300, SEARS TOWER, CHICAGO, IL, 60606, US

(73) Assignee: APPLIED RESEARCH SYSTEMS ARS HOLDING N.V., Curacao (NL)

(21) Appl. No.: 10/579,286

(22) PCT Filed: Dec. 6, 2004
(86) PCT No.: PCT/US04/40547

§ 371 Date: Jan. 12, 2007

Related U.S. Application Data
(60) Provisional application No. 60/526,930, filed on Dec. 4, 2003.

(51) Int. Cl. A61K 31/00; C07K 16/00; G01N 33/567

(52) U.S. Cl. 514/1; 435/7.2; 435/7.21; 530/387.1

(57) ABSTRACT

The present invention relates to cell-based assays useful for screening for modulators, such as inhibitors, of activated mutant KIT tyrosine kinase receptors, which are associated with mast cell-related disorders, such as mastocytosis and various types of cancer. The invention further provides for the treatment of mast cell-related disorders with an inhibitor identified by the screening method.

### FIELD OF THE INVENTION

The present invention relates to a cell-based assay useful for screening for inhibitors of activated mutant KIT tyrosine kinase receptors. Mutated KIT receptors are involved in mast cell-related disorders, such as mastocytosis, and numerous types of cancer. The invention further contemplates treatment of mast cell related disorders with an inhibitor identified by the screening method.

### BACKGROUND OF THE INVENTION

KIT tyrosine kinase receptor is a type III transmembrane receptor found primarily on cells of the hematopoietic lineage, e.g. bone marrow cells, mast cells, and T cells, but is also detectable in melanocytes, testis, vascular endothelial cells, interstitial cells of Cajal, astrocytes, renal tubules, breast epithelial cells, and cells of the sweat glands (Ashman, L., Int. J. Biochem. Cell. Bio. 31:1037-51, 1999). KIT receptor is a key molecule in regulating the growth and survival of mast cells (Longley, Jr. et al., Proc. Natl. Acad. Sci. USA 96:1609-1614, 1999). The KIT receptor comprises an extracellular domain containing five immunoglobulin domains, a transmembrane domain, and an intracellular region containing a split kinase domain. One lobe of a kinase domain acts as an ATP binding domain while the other functions as a phosphotransferase domain comprising a kinase activation loop.

The interaction of KIT with its ligand, stem cell factor (SCF) (also known as steel, mast cell growth factor, or KIT ligand), via the extracellular domain results in receptor dimerization. The KIT-dimer auto-phosphorylates at specific tyrosine residues in the intracellular region due to transphosphorylation within the dimer. KIT phosphorylation activates the receptor and triggers a cascade of downstream signaling events involved in a variety of physiological processes, including cellular proliferation (Longley et al, supra).

Irregular KIT activation has been implicated in the development of both spontaneous and familial mastocytosis. Mastocytosis is characterized by excess proliferation of mast cells, distributed in a predictable pattern throughout the skin (e.g., urticaria pigmentosa), bone marrow, gastrointestinal tract, lymph nodes, liver and spleen (Brockow et al., Curr. Opin. Allergy Clin. Immunol. 1:449-54. 2001). Mastocytosis is classified as either familial or sporadic, the latter being further subdivided into either cutaneous or systemic. Systemic mastocytosis is still further classified into indolent (chronic) mastocytosis and aggressive mastocytosis. Types of mastocytosis also emerge which have an associated hematologic disorder (AHD) (Brockow et al., supra). Many cases of pediatric mastocytosis are associated with constitutively activated KIT receptors. Leukemias associated with mastocytosis include mast cell leukemia.

The majority of mastocytosis and related disorders are caused by spontaneous somatic mutation in the KIT receptor, creating a constitutively phosphorylated, active receptor that induces increased mast cell proliferation. (Brockow, supra). Several KIT mutations identified to date which result in an activated receptor are located in the kinase domains, particularly in the KIT kinase activation loop. The activating mutation induces dimerization of the receptor without stimulation by SCF and causes aberrant cell proliferation and other cellular activity, such as cytokine secretion.

Presently, there is no cure for mastocytosis, and few candidate therapies exist. A significant drawback to these therapies is the non-specific inhibition of many cellular tyrosine kinases in the cells targeted by the treatment. For instance, two promising kinase inhibitor therapeutics for treating mastocytosis were originally used to inhibit other kinases, such as platelet-derived growth factor receptor (PDGF-R), vascular endothelial growth factor receptor (VEGFR), or the Bcr/Ab1 mutation. These potential therapeutics proved ineffective at treating all forms of mastocytosis.

Previous treatments of mast cells having mutant KIT receptors with indolinone derived kinase inhibitors have proven partially successful. (Ma et al., J. Invest Dermatol. 114:392-4, 2000). The majority of indolinones inhibit SCF-activated wild-type KIT receptor, but do not inhibit phosphorylation of all juxtamembrane and activation loop mutations (Ma et al., supra). Three out of five compounds inhibited juxtamembrane mutations in C2 canine mast cells, i.e. an insertion mutation in KIT, while only SU6577, a PDGF-R and VEGF-R inhibitor, decreased tyrosine phosphorylation in P815 murine mast cells expressing the KITD814V activation loop mutation. This result indicates that each KIT mutation is unique and one KIT inhibitor is not universally effective.

Several factors contribute to the difficulty in identifying potential inhibitors of KIT receptor in high-throughput screens and in high-content assays. For example, in vitro, test tube based assays analyzing KIT receptor activation require purified protein in amounts sufficient to measure KIT receptor phosphorylation using various biochemical methods. To obtain purified protein, the KIT receptor must be expressed in a recombinant system such as bacterial, yeast or even mammalian cells, with the expressed protein subsequently purified from these sources.

Most mutant KIT receptors expressed in these recombinant systems are toxic to the host cells and cannot be produced in amounts sufficient to perform the assays. Additionally, KIT receptor produced in yeast or bacteria is typically inactive, possibly due to lack of proper post-translational modification to carry out normal protein function.

Cell-based assays are slowly being developed by companies to measure kinase activity. Phospho-specific antibodies are being produced to detect specific target protein phosphorylation such as phospho-MAPK or phospho-HER2 kinase (Cell Signaling Technology, Beverly, Mass.) and phospho-KIT (pY823, Biosource, Inc.). Biosource, Inc. recently developed an antibody to the phosphorylated tyrosine 823 residue of KIT. However, this antibody has been described as useful in Western blot and in vitro kinase assays only, not for a cell-based assay of KIT receptor activity.

Cell-based assays have been developed to detect the activity of a few cell-signaling proteins, but these analyses rely on the translocation of the signaling protein from the cytoplasm to the nucleus (Cellomics, Inc., Pittsburgh, Pa.), a noticeable change in protein activation. Activation by protein phosphorylation involves a subtle change that can be difficult to detect in a complex cellular background without a discriminatory advantage provided by, e.g., a highly sensitive, molecule-specific assay.

Cell-based assays that assess tyrosine phosphorylation have been particularly difficult to develop. Because tyrosine phosphorylation is a common downstream event in numerous signaling cascades, assessment of a single target protein's phosphorylation state is confounded by detection of background phosphorylation. Further, intracellular assays require permeabilization of cells which increases the non-specific signal due to a certain degree of cell death or cell lysis resulting from the permeabilization process. Development of cell-based assays for detecting KIT receptor activation have been hampered by these difficulties.

Current cell-based assays indirectly measure kinase activity in terms of cell proliferation. For instance, an assay used to assess KIT receptor activity described activation/inhibition of SCF-stimulated, wild-type receptor in terms of cell proliferation (Heinrich et al., Blood 96:925-32, 2000). This type of assay does not measure the actual activity of the receptor itself, with the reliability of the measure compromised by the variety of additional cellular influences on proliferation, and only measures cell activation non-specifically.

Thus, there exists a need in the art to develop assays suitable for high-throughput screening for inhibitors of KIT tyrosine kinase receptors and to develop new and improved therapeutics for the treatment of mast cell disorders. Moreover, there exists a need in the art for a method for preventing and diagnosing a variety of mast cell disorders affecting all animals, including humans, which collectively contribute to high health costs.

### SUMMARY OF THE INVENTION

The present invention addresses at least one of the aforementioned needs in the art relating to the treatment and regulation of mast cell disorders, by providing a method for screening candidate compounds and identifying modulators, such as inhibitors, of activated KIT tyrosine kinase receptors, which is correlated with the development of mast cell disorders. The present invention provides a sensitive assay for the identification of inhibitors of KIT receptors, including constitutively active KIT receptors, useful for the treatment of mast cell disorders such as mastocytosis, mast cell leukemia, acute myeloid leukemia, and chronic myelogenous leukemia. Moreover, the present invention provides an advantage over traditional assays by providing a cost-effective, cell-based assay to directly assess the effects of an inhibitor on KIT tyrosine kinase receptor activity.

The present invention provides a method of screening for an inhibitor of an active KIT tyrosine kinase receptor in a cell comprising: (a) contacting a cell comprising an active KIT tyrosine kinase receptor with a candidate inhibitor; and (b) detecting KIT activity by using a phosphotyrosine-specific antibody to determine the amount of KIT tyrosine phosphorylation in the presence and in the absence of the inhibitor, wherein a decrease in KIT tyrosine phosphorylation in the presence of the candidate inhibitor in comparison to the KIT tyrosine phosphorylation in its absence identifies the candidate inhibitor as a KIT inhibitor.

In one embodiment, the active KIT receptor is activated by contact with its ligand. In another embodiment, the KIT tyrosine kinase receptor is constitutively active. As used herein, “constitutively active” means the receptor is phosphorylated in the absence of ligand stimulation, as a result of a mutation in the KIT receptor. In an embodiment, the constitutively active KIT tyrosine kinase receptor has a mutation in a tyrosine kinase domain of the receptor. The mutation in the tyrosine kinase domain is in either the first or second kinase domain of the KIT receptor. When the mutation is in the first kinase domain, it is selected from the group consisting of exon 13 mutations and substitution mutation K642E. In one embodiment, the mutation in a tyrosine kinase domain of the KIT receptor is in the activation loop of the KIT tyrosine kinase domain. In one embodiment, the activation loop domain mutation is selected from the group consisting of a mutation at residue 816 of SEQ ID NO:2, particularly D816V, D816H, D816F, D816N, and D816Y, a substitution mutation D820G in SEQ ID NO:2, and a substitution mutation V825A in SEQ ID NO:2. In one embodiment, the substitution mutation comprises a valine substitution at residue 816.

In another embodiment, the constitutively active KIT tyrosine kinase receptor has a mutation in the juxtamembrane domain. The juxtamembrane domain mutation is selected from the group consisting of a mutation in exon 11 of SEQ ID NO:2, a deletion of amino acids 550-558 (ΔK550-558) of SEQ ID NO:2, and a glycine substitution for valine at residue 560 (V560G). In other embodiments, the constitutively active KIT receptor contains a mutation in the extracellular domain. In one embodiment, the extracellular domain mutation is selected from the group consisting of a mutation in exon 9 and a substitution mutation AY502-503 in SEQ ID NO:2.

Some embodiments include a KIT tyrosine kinase receptor which comprises an amino acid sequence selected from the group consisting of SEQ ID NOS:2, 4 and 6. In one specific embodiment, the KIT tyrosine kinase receptor comprises the amino acid sequence set forth in SEQ ID NO:2.

In one embodiment, the contacting step is performed by incubating the candidate inhibitor with the cells in a suitable buffer. In some embodiments, the method provides that the contacting step is performed wherein the cell comprising the active KIT tyrosine kinase receptor is bound to a solid support or free in solution. In one embodiment, the cell comprising the KIT receptor is bound to a solid support. It is contemplated that the solid support is a plastic or glass plate appropriate for tissue culture purposes and use in microscopy. It is further contemplated that the solid support is selected from the group consisting of plastic or glass dishes, cover slips, clear bottom microtiter plates, and round bottom microtiter plates. In a related embodiment, the cell comprising the active KIT receptor is free in solution. The solution may be any buffered solution appropriate for culturing cells or staining cells as described herein.

In some embodiments, the method provides that when the cell comprising active KIT receptor is bound to a solid support or free in solution, analyses are made in addition to detecting the KIT activation and phosphorylation. These analyses comprise such assays as detecting cellular morphology, cytoskeletal rearrangement, or nuclear staining of the cell in the presence and in the absence of the candidate inhibitor. It is contemplated that detection of cellular morphology, cytoskeletal rearrangement or nuclear staining is performed using fluorescent techniques such as fluorescent microscopy or flow cytometry. It is further contemplated that the detection for cellular morphology, cytoskeletal rearrangement or nuclear staining is performed using contrast microscopy, such as bright field staining or hematoxilyn/eosin staining.

The method of the invention also provides for the detection of KIT receptor activity using a phosphotyrosine-specific antibody. It is contemplated that the phosphotyrosine-specific antibody is selected from the group consisting of a monoclonal antibody, a polyclonal antibody, a chimeric antibody, a humanized antibody, a single-chain antibody, and an antibody fragment. In one embodiment the antibody is a polyclonal antibody. In another embodiment the phosphotyrosine-specific antibody is pY823. In a related embodiment, the phosphotyrosine-specific antibody binds to an auto-phosphorylation site of the KIT tyrosine kinase receptor.

It is further contemplated that the phosphotyrosine-specific antibody is detectably labeled. In some embodiments, the detectable label is a fluorophore, part of a binding partner pair, or a radiolabel. The fluorophore contemplated for use includes any fluorophore or colorimetric label suitable for conjugation to an antibody and detectable using methods well-known in the art. For instance, the fluorophore can be fluoroisothiocyanate, phycoerythrin, APC, PerCP, AlexaFluor molecules, Cy3, Cy5, Texas red, and phalloidin. In another embodiment, the detectable label comprises one half of a binding partner pair. The invention contemplates that the binding partner pair is selected from the group consisting of biotin/streptavidin, His₆ peptide tags/anti-His₆-tag antibodies, biotin/anti-biotin molecules, and fluorophore/anti-fluorophore molecules, wherein the second binding partner comprises a label detectable through enzyme/substrate labeling such as horse radish peroxidase, alkaline phosphatase, or other suitable enzyme/substrate pair. It is further contemplated that the second binding partner is conjugated to a radiolabel as described below. In an additional embodiment, the phosphotyrosine antibody comprises a radiolabel, wherein the radiolabel is selected from the group consisting of tritiated thymidine (³H), Europium³⁺ (Eu), and ³²P. In some embodiments, the detecting step comprises detection by flow cytometry.

In some embodiments of the invention, the active KIT tyrosine kinase receptor is expressed from a heterologous vector. In one embodiment, the active KIT receptor is produced using genetic engineering as described herein. The KIT receptor polynucleotide of SEQ ID NO:1 may be manipulated to contain a substitution mutation, a deletion mutation, or an insertion mutation which results in a KIT receptor that is constitutively active, and subsequently inserted into a suitable heterologous expression vector. It is contemplated that the recombinant KIT receptor containing heterologous vector is transfected into an appropriate host cell, wherein the transfected host cell expresses the recombinant KIT receptor polypeptide.

In some embodiments, the active KIT tyrosine kinase receptor is endogenous to the cell. In one specific embodiment, the cell that endogenously expresses active KIT receptor is a cell line, wherein the cell line is selected from the group consisting of human mast cell lines (e.g. TF-1 and HMC-1), mast cell lines from other species, (e.g. P815, FMA3, RBL-2H3, and C2), c-Kit expressing cell lines (e.g. NCI-H187, NCI-H378, and NCI-H526), and germ cell tumor/seminoma cell lines. In another embodiment, the cell comprising the active KIT receptor is isolated from a tumor, wherein the tumor is selected from the group consisting of a mast cell leukemia, mast cell sarcoma, a germ cell tumor, a gastrointestinal stromal tumor, an acute myeloid leukemia (AML), a chronic myeloid leukemia (CML), a chronic myelomonocytic leukemia (CMML), a sinonasal lymphoma, an ovarian tumor, a breast tumor, a small lung cell carcinoma, a neuroblastoma, and a melanoma.

The invention further provides a kit for screening for an inhibitor of active KIT tyrosine kinase receptor, wherein the kit comprises a phosphotyrosine antibody and instructions for performing a screen for the inhibitor.

Also provided is an inhibitor identified by a method of the invention. It is contemplated that the invention provides a pharmaceutical composition comprising the inhibitor identified by the method of the invention and a pharmaceutically acceptable diluent, adjuvant, or carrier. Acceptable diluents and carriers and methods of formulating a pharmaceutical composition are described herein.

The invention also provides a method of treating a condition selected from the group consisting of mastocytosis, mast cell leukemia, mast cell sarcoma, a germ cell tumor, a gastrointestinal stromal tumor, an acute myeloid leukemia (AML), a chronic myeloid leukemia (CML), a chronic myelomonocytic leukemia (CMML), a sinonasal lymphoma, an ovarian tumor, a breast tumor, a small lung cell carcinoma, a neuroblastoma, and a melanoma and comprising administering a pharmaceutically acceptable dose of the inhibitor identified in the screening method of the invention.

Preferably, an inhibitor of KIT receptor is administered to a mammalian subject, and more preferably the mammalian subject is human. The invention contemplates that the condition being treated is characterized by aberrant growth or proliferation of cells expressing a mutant KIT receptor. In one embodiment the cells are mast cells. The administration of the inhibitor beneficially alters the aberrant growth or proliferation, e.g., by correcting it, or reducing its severity, or reducing its deleterious symptoms or effects.

For example, in one variation, the animal has a cancer, especially a cancerous tumor resulting from aberrant mast cell proliferation. An inhibitor of an activated KIT receptor is identified with the expectation that it will decrease growth, migration, or proliferation of the mast cells, and thereby retard the growth of the tumor. It is contemplated that the inhibitor identified by the invention is administered in conjunction with chemotherapeutic agents, to further accelerate the tumor regression and decrease aberrantly proliferating mast cells. Exemplary chemotherapeutic agents include anti-metabolites such as 5-FU, gemcitabine, cytarabine, methotrexate, hydroxyurea, and 6-thioguanine; DNA-damaging agents; cytokines; covalent DNA-binding drugs such as platinum containing complexes; topoisomerase inhibitors such as camptothecin, irinotecan, topotecan, and etoposide; anti-tumor antibiotics such as the doxorubicin, actinomycin-C, daunorubicin, and bleomycin; differentiation agents; alkylating agents; methylating agents; nitrogen mustards; and radiation sources, optionally combined with radiosensitizers and/or photosensitizers; or other commonly used therapeutic agents.

Any administration route and regimen known in the art may be used in the treatment methods according to the invention. Administration of the inhibitor is determined by the administering physician and may be based on one or more variables known in the art and typically relied on by practitioners, such as the weight of the subject treated. For example, the amount of inhibitor given will vary according to the size of the individual to whom the therapy is being administered (on a mg inhibitor/kg body weight basis), and may range from about 50 mg/kg day, 75 mg/kg day, 100 mg/kg day, 150 mg/kg day, 200 mg/kg day, 250 mg/kg day, 500 mg/kg day or 1000 mg/kg day.

The invention also contemplates a method for designing a treatment regimen for a patient with a mast cell disorder comprising: (a) isolating a cell from the patient, wherein the cell comprises an active KIT tyrosine kinase receptor; (b) contacting the cell with a KIT inhibitor identified as described above; (c) detecting KIT activity in the cell using a phosphotyrosine-specific antibody to determine the amount of KIT tyrosine phosphorylation in the presence and in the absence of the inhibitor; and, (d) designing a treatment regimen for the patient which includes administration of the KIT inhibitor that specifically inhibits KIT activity in the patient.

In one aspect, the treatment regimen is designed to target the particular mast cell disorder or condition exhibited by the patient. The mast cell disorder is selected from those previously described above. In one embodiment, the method involves determining the mast cell inhibitor that exhibits the greatest degree of KIT receptor inactivation in the patient having a mast cell disorder, wherein the determination is based on KIT receptor inhibition of cells isolated from the patient. In one embodiment, the cells are isolated from fluid or tissue samples from humans or animals. Such samples are obtained by methods well known in the art. Exemplary biological fluid samples include blood, cerebrospinal fluid, urine, and saliva. Exemplary tissue samples include normal tissue samples, tumors, and biopsies thereof. It is contemplated that the cells isolated from the patient are analyzed using a screening method as described above, wherein the cells are contacted with an inhibitor and KIT receptor activation is monitored using a phosphotyrosine antibody as described.

It is further contemplated that once an inhibitor is identified that is an inhibitor of the patient's specific mast cell disorder, a treatment regimen is designed wherein the identified inhibitor is administered to the patient being treated. In one embodiment, the inhibitor is administered in a pharmaceutically acceptable carrier in an amount effective to inhibit the mast cell disorder. In a related embodiment, the inhibitor is administered in conjunction with other chemotherapeutics to provide a synergistic effect and accelerate tumor regression or decrease mast cell proliferation in the patient.

Numerous additional aspects and advantages of the invention will become apparent to those skilled in the art upon consideration of the following detailed description of the invention which describes presently preferred embodiments thereof.

### DETAILED DESCRIPTION

The invention addresses a need in the art by providing a sensitive cell-based assay that specifically detects activated KIT tyrosine phosphorylation. The invention provides a cost effective assay that does not require substantial amounts of purified, in vitro active protein. The invention further provides a sensitive assay for the identification of inhibitors of KIT receptors useful for the treatment of mast cell disorders such as mastocytosis, and mast cell leukemia, acute myeloid leukemia, and chronic myelogenous leukemia.

The invention provides discriminatory and sensitive methods of screening for inhibitors of KIT tyrosine kinase receptors. The methods include use of a cell-based assay to detect intracellular activation and tyrosine phosphorylation of a receptor in the presence and in the absence of a candidate inhibitor. The invention provides a means for assessing direct effects of inhibitor on the KIT protein rather than simply detecting a non-specific response to a candidate inhibitor, such as measuring cell proliferation or cell death. The cell-based assay provides the benefit of functioning with a reduced quantity of KIT receptor as compared to the quantities required for standard in vitro, test tube assays. Further, the cell-based assays do not require the costly, cumbersome, and time-consuming process of purifying a KIT receptor. Detecting inhibitors of KIT receptor activation enables the development of new therapeutics for the treatment of such disorders as chronic mastocytosis, aggressive mastocytosis, systemic mastocytosis, cutaneous mastocytosis, sporadic mastocytosis, familial mastocytosis, acute myeloid leukemia, chronic myeloid leukemia, chronic myelomonocytic leukemia, and any other disorder characterized by aberrant growth or proliferation of mast cells.

To facilitate a more thorough understanding of the invention, the following term definitions are provided.

An “active” or “activated KIT” is a KIT tyrosine kinase receptor in dimerized form that exhibits tyrosine phosphorylation. The KIT receptor may be activated either through stimulation with its ligand, SCF, or it may be constitutively active as a result of a mutation.

A “mutant KIT” as used herein is a KIT receptor that differs in sequence from the wild-type KIT by amino acid deletion, insertion, or substitution, and which exhibits a constitutively active phenotype. The mutation may be in the extracellular domain or the intracellular domain of the KIT receptor.

A “cell comprising an active KIT” is any cell line or cell isolated from a subject that expresses a wild-type or mutant KIT, regardless of whether that expression occurs naturally (i.e. native expression under at least one set of conditions expected to be found in nature) or is genetically engineered in whole or part.

A “candidate inhibitor” is a compound or molecule that may inhibit activation of at least one KIT receptor and that can be subjected to a method of the invention for assessing the ability of a compound to inhibit KIT receptor activation through its ligand or to inhibit a constitutively active KIT receptor.

A “phospho-specific antibody” is an antibody that specifically binds to a phosphorylated compound such as a phosphorylated protein. A phospho-specific antibody may specifically recognize a binding site comprising a phosphorylated serine, threonine or tyrosine. It should be understood that reference to phospho-specific antibody, as used herein, typically refers to phosphotyrosine-specific antibody, as would be apparent from usage of the term in context.

“Autophosphorylation” is the addition of a phosphate to a protein kinase using its own enzymatic activity, without direct participation by another molecule. Autophosphorylation of the KIT receptor can be caused by stimulation through a ligand or due to a mutation in the KIT receptor.

By “detecting cellular morphology cytoskeletal rearrangement, or nuclear staining” is meant monitoring, in the presence and absence of the candidate inhibitor; i) cell membrane integrity and cell shape; ii) cytoskeletal composition, fiber assembly, and shape, and; iii) nuclear DNA composition in the nucleus, preferably looking at changes in apoptotic or proliferating cellular nuclei, respectively.

A “heterologous vector” is a vector used to express a nucleic acid or protein not naturally expressed in a host cell or not expressed at sufficient levels for purification or detection of the encoded protein. Particular vectors useful for the invention are discussed in detail.

By “endogenous” is meant that a nucleic acid or protein is naturally expressed in a host cell, which can be either a cell line or a cell isolated from a subject.

The term “selectivity,” when used herein to describe inhibitors, refers to the ability of a KIT inhibitor to inhibit one protein activity (e.g., KIT phosphorylation) with minimal effects on the interaction of another protein activity or protein-protein interaction.

The term “hybrid hybridoma” is used to describe the productive fusion of two B cell hybridomas.

The term “substantially similar” refers both to nucleotide and amino acid sequences, for example a mutant sequence, that varies from a reference sequence by one or more substitutions, deletions, or additions, the net effect of which does not result in an adverse functional dissimilarity between the reference and subject sequences. The variation may be 1 nucleotide or amino acid, up to 5 nucleotides or amino acids, up to 10 nucleotides or amino acids, up to 20 nucleotides or amino acids, up to 50 nucleotides or amino acids, or up to 150 nucleotides or amino acids.

A. Mast Cell Disorders and KIT Receptor Mutations

Human KIT receptor (Genbank Accession No. NM_{—}000222) is a protein of 976 amino acids (SEQ ID NO: 2) comprising a signal peptide from residues 1-22, immunoglobulin-like regions from approximately residues 43-112, residues 224-297, and residues 320-410, and split tyrosine kinase domains from amino acids 589-694 and amino acids 771-924. The first lobe (residues 589-694) comprises the ATP binding domain while the second lobe is defined as the phosphotransferase domain and contains a kinase activation loop (Féger et al., Int. Arch. Allergy Immunol. 127:110-14, 2002). Exon 11 of the KIT protein is an important region termed the juxtamembrane domain, which is important in receptor activity and functions as an anti-dimerization domain to regulate proper receptor dimerization. KIT receptor homologs exist in most other mammalian species, including mouse (Genbank Accession No. NM_{—}021099, SEQ ID NO: 3 and 4) and rat (Genbank Accession No. NM_{—}022264; SEQ ID NO: 5 and 6).

The interaction of KIT receptor with its ligand drives mast cell proliferation and differentiation (Féger et al., supra). Mutations in KIT receptor that cause dysfunction of the receptor often result in mastocytosis or a related mast cell disorder. The majority of KIT mutations associated with the onset of mastocytosis are somatic mutations arising spontaneously in the juxtamembrane domain or in either of the kinase domains. For example, a valine for glycine substitution at residue 560 (V560G), or deletion of 9 amino acid residues beginning with the lysine at residue 550 (ΔK550-558), both in the juxtamembrane domain, have been associated with gastrointestinal stromal tumors. A mutation in the juxtamembrane domain has also been identified in patients with sinonasal natural killer/T cell lymphoma (Heinrich et al., J. Clin. Oncol. 20:1692-1703, 2002). Mutations in the juxtamembrane domain, designated “regulatory mutations,” disrupt the regulatory (e.g. inhibitory) function of this KIT receptor region, and result in phosphorylation of the KIT receptor (Longley et al., Leukemia Res. 25:571-76, 2001). The KIT inhibitor Gleevec (Novartis AG, Parsippany, N.J.) has been shown to inhibit constitutively active KIT mutants exhibiting mutations in the juxtamembrane domain (Frost et al., Mol. Cancer Ther. 1:1115-24, 2002).

Mutations in the kinase domains are associated with mastocytosis and leukemias. Substitution mutations located at residue 816 in the KIT receptor tyrosine kinase domain, and more specifically in the kinase activation loop, have been associated with the majority of cases of adult sporadic mastocytosis. Mastocytosis associated mutations include wild-type aspartic acid (D816) substituted with valine (D816V), phenylalanine (D816F) and tyrosine (D816Y). A histidine substitution at 816 (D816H) has been identified in patients with germ cell tumors, such as seminoma. A substitution of asparagine for aspartic acid (D816N) was detected in patients with sinonasal tumors. Additionally, KIT receptor mutations D816Y and D816V have been found in patients with AML. Analogous mutations are found in mouse KIT receptor at residue 814 (D814Y) and rat KIT receptor at residue 817 (D817Y) (Féger et al., supra). These analogous mutations all lie in the activation loop domain and are designated “activating mutations,” due to the ligand-independent phosphorylation induced by these mutations. Other activating mutations include K642E found in gastrointestinal stromal tumors (GISTs), a mutation in extracellular exon 9 and intracellular exon 17, and potentially a mutation at D820G (Heinrich et al., J. Clin. Oncol. 20: 1692-1703. 2002).

While some specific treatments for mastocytosis exist, e.g. gastrocrom, treatment regimens for mastocytosis typically employ non-specific treatment regimens used in other proliferative disorders (e.g., histamine receptor blockers, prostaglandin blockers, steroids (severe cases)), resulting in incomplete treatment or treatments which are not effective in advanced forms of mastocytosis. For example, a patient treated with IFN-α_{2b} and the immunosuppressant prednisolone demonstrated incomplete tumor excision (Brockow et al, supra). Mastocytosis patients may be treated with the purine nucleoside cladribrine, which exhibited improved effects over IFN-α_{2b} treatment. Compounds that specifically inhibit KIT activity have been contemplated and tried in vitro, but no successful method of specifically treating mastocytosis has been developed. Thus, there still exists a need in the art to provide assays which identify compounds useful for the treatment of mastocytosis by KIT tyrosine kinase inhibitors.

B. Polynucleotides for Use in the Method of the Invention

Polynucleotides for use in the method of the invention include DNA (genomic, complementary, amplified, or synthetic) and RNA, as well as polynucleotide mimetics that, while chemically distinct from naturally occurring polynucleotides, encode a KIT receptor polypeptide that can be expressed in a manner similar to a KIT receptor polypeptide encoded by a polynucleotide of the invention. Polynucleotides for use in the invention include, but are not limited to, a purified and isolated polynucleotide encoding a KIT receptor polypeptide (SEQ ID NO:2), or a fragment thereof encoded by the polynucleotide set out in SEQ ID NO:1. In various aspects, the invention provides for use of polynucleotides comprising sequences as set out in SEQ ID NO:1, or variants thereof, that encode a KIT receptor. The polynucleotides useful in the invention also include, but are not limited to, a polynucleotide comprising a polypeptide-coding region that specifically hybridizes under stringent conditions to (a) the complement of SEQ ID NO:1, (b) a polynucleotide encoding a polypeptide selected from the group consisting of SEQ ID NO: 2 (c) a polynucleotide encoding a polypeptide which is substantially similar to a KIT receptor encoded by a polynucleotide of the invention, (d) polynucleotides encoding variant polypeptides which possess at least one biological activity of KIT receptor, and (e) a polynucleotide which encodes a homolog of any of the polypeptides recited above, wherein the polypeptide possesses the KIT receptor activity.

The term “stringent” as used herein refers to the degree of rigor of the physico-chemical conditions (e.g. temperature, salt, pH) of nucleic acid hybridization. Highly stringent hybridization conditions include a final wash in 0.1×SSC/0.1% SDS at 65° C., or equivalent conditions as would be known in the art. See e.g. Sambrook, et al., in Molecular Cloning: A Laboratory Manual, Second Edition, Cold Spring Harbor, N.Y. (1989). Moderately stringent conditions involve a final wash in 0.2×SSC/0.1% SDS at 42° C. or equivalent conditions. In instances of hybridization of oligonucleotides that encode a KIT receptor, or a probe that can be used to specifically identify a polynucleotide encoding such a KIT receptor, exemplary stringent hybridization conditions include washing in 6×SSC/0.05% sodium pyrophosphate at 37° C. (for 14-base oligonucleotides), 48° C. (for 17-base oligonucleotides), 55° C. (for 20-base oligonucleotides), and 60° C. (for 23-base oligonucleotides). Included within the scope of the nucleic acids useful in the method of the invention are nucleic acids comprising fragments of a polynucleotide encoding a full-length KIT receptor and nucleic acids that specifically hybridize under stringent conditions to any such polynucleotide or fragment thereof of the nucleotide sequences of the invention, or complements thereof, wherein such fragments and nucleic acids preferably encode a peptide that retains at least one biological activity of a KIT receptor. The fragment and nucleic acids are preferably greater than about 10 nucleotides, and more preferably greater than 17 nucleotides. Fragments of about 15, about 17, or about 20 nucleotides or more that are selective for (i.e., specifically hybridize to) any one of the polynucleotides of the invention are contemplated.

Polynucleotides according to the invention include those that have, e.g., at least about 70%, at least about 75%, at least about 80%, 81%, 82%, 83%, 84%, 85%, 86%, 87%, 88%, or 89%, preferably at least about 90%, 91%, 92%, 93%, or 94% and more preferably at least about 95%, 96%, 97%, 98%, 99%, 99.5%, or 99.9% sequence identity to a polynucleotide comprising a sequence expressly set forth herein that retains biological activity (e.g., encodes a peptide exhibiting immunological, catalytic and/or respective activity of a KIT receptor).

“Variant” polynucleotides contemplated for use in the invention include naturally occurring polynucleotides as well as chemically altered polynucleotides. Naturally occurring polynucleotide variants of the invention are those that (i) are found in nature, e.g., in related mammalian species, (ii) are related to a polynucleotide of the invention through chemical similarity as described herein, and (iii) encode a polypeptide that exhibits at least one KIT receptor activity. Exemplary variant polynucleotides include polynucleotides set out in SEQ ID NO: 3 and SEQ ID NO: 5. Variants of this type and others are identified using the hybridization and probe techniques as described above.

Chemically altered, or synthetic, polynucleotide sequence variants are those that are not found in nature, and variants of this type may be prepared by methods known in the art. For example, nucleotide changes may be introduced into a naturally occurring polynucleotide to effect changes in the encoded polypeptide sequence. There are at least two variables to be considered in construction of amino acid sequence variants—the location of the mutation and the nature of the mutation. These nucleic acid alterations can be made at sites that differ in the nucleic acids from different species (variable positions) or in highly conserved regions (constant regions). Sites at such locations will typically be modified in series, e.g. by substituting first with conservative choices (e.g., hydrophobic amino acid substituted for a non-identical hydrophobic amino acid) and then with more dissimilar choices (e.g., hydrophobic amino acid substituted for a charged amino acid), and then deletions or insertions may be made at the target site.

“Conservative” amino acid substitutions may be made on the basis of similarity in polarity, charge, solubility, hydrophobicity, hydrophilicity, and/or the amphipathic nature of the residues involved. For example, nonpolar (hydrophobic) amino acids include alanine (Ala, A), leucine (Leu, L), isoleucine (Ile, I), valine (Val, V), proline (Pro, P), phenylalanine (Phe, F), tryptophan (Trp, W), and methionine (Met, M); polar neutral amino acids include glycine (Gly, G), serine (Ser, S), threonine (Thr, T), cysteine (Cys, C), tyrosine (Tyr, Y), asparagine (Asn, N), and glutamine (Gln, Q); positively charged (basic) amino acids include arginine (Arg, R), lysine (Lys, K), and histidine (His, H); and negatively charged (acidic) amino acids include aspartic acid (Asp, D) and glutamic acid (Glu, E). “Insertions” or “deletions” are preferably in the range of about 1 to 20 amino acids, more preferably 1 to 10 amino acids. The variation allowed may be experimentally determined by routine methods well known to those of skill in the art.

Due to the inherent degeneracy of the genetic code, other DNA sequences may encode the same amino acid sequence, and nucleic acids comprising any of these other (i.e., degenerate) sequences are embraced by the invention. These “degenerate variants” differ from a nucleic acid fragment of the present invention by nucleotide sequence but, due to the degeneracy of the genetic code, encode an identical polypeptide sequence. Various codon substitutions, such as silent changes which produce various restriction sites, may be introduced to optimize cloning into a plasmid or viral vector or expression in a particular prokaryotic or eukaryotic system. Such nucleic acids include those which are capable of hybridizing to a nucleic acid as described herein under stringent conditions, preferably, highly stringent conditions.

The term “variant” (or “analog”) therefore refers to any polypeptide differing from naturally occurring polypeptides by amino acid insertions, deletions, and substitutions. These variants or analogs may be constructed using, e.g., recombinant DNA techniques. Guidance in determining which amino acid residues may be replaced, added or deleted without abolishing activities of interest, may be found by comparing the sequence of the particular polypeptide with that of homologous peptides and minimizing the number of amino acid sequence changes made in regions of high homology (conserved regions) or by replacing amino acids in conserved regions in a manner that shifts a given sequence closer to an art recognized consensus sequence. Variants may be produced using any method known in the are, including site-directed mutagenesis.

The polynucleotides useful in the invention additionally include the complement of any of the polynucleotides recited above. Complementary sequences of this type are particularly useful in the identification of related sequences as described herein, as well as serving as template polynucleotides from which synthetic variants of the invention can be prepared. For example, such synthetic variants can be generated using polymerase chain reaction (PCR) under optimized standard conditions.

The invention further provides for use of “chimeric polynucleotides” encoding proteins comprising a fusion of KIT receptor and a heterologous amino acid sequence wherein the chimeric polynucleotide encodes a polypeptide that retains at least one biological activity of KIT receptor. As used herein, a “heterologous” polynucleotide comprises a polypeptide coding region linked in proper reading frame (“in-frame”), via techniques described herein or otherwise known in the art, to a second protein coding sequence, wherein the first, heterologous polypeptide coding region is not naturally associated with (adjacent to) the second polypeptide coding sequence in nature. Specifically contemplated are chimeric polynucleotides (and “chimeric polypeptides” encoded by the polynucleotides) comprising a first, heterologous polynucleotide described previously which encodes a polypeptide operably linked to a second polynucleotide of the invention. Within the chimeric polynucleotides, the term “operatively linked” is intended to indicate that the heterologous polynucleotide and the KIT receptor polynucleotide are attached in-frame with one another so that the expressed polypeptide includes both encoded sequences.

Chimeric polynucleotide sequences comprising KIT receptor may be used to generate recombinant DNA molecules that direct the expression of that nucleic acid in appropriate host cells. A heterologous polynucleotide according to the invention can be joined to any of a variety of other nucleotide sequences by well-established recombinant DNA techniques (see Sambrook J et al. supra).

The invention further provides chimeric polynucleotides inserted into a vector, such as an expression vector or a heterologous vector for the purpose of expressing an encoded polypeptide. Expression vectors comprise a capacity to incorporate a coding region and the necessary elements required for expression of that coding region in at least one host cell, as would be known in the art. typically, such vectors contain at a minimum a promoter, properly oriented to facilitate RNA expression of the coding region. Suitable expression vectors and host cells are known in the art. Useful vectors include, e.g., plasmids, cosmids, viruses such as lambda phage and its derivatives, phagemids, artificial chromosomes, and the like, that are well known in the art. In general, the vector contains an origin of replication functional in at least one organism, convenient restriction endonuclease sites, and a selectable marker for the host cell. Vectors according to the invention include expression vectors, replication vectors, probe generation vectors, and sequencing vectors.

In the case of a vector comprising a KIT tyrosine kinase receptor coding region, the vector may further comprise regulatory sequences, including, for example, a promoter, operably linked to the heterologous nucleotide sequence. Large numbers of suitable vectors (many of which include endogenous regulatory DNA elements) are known to those of skill in the art and are commercially available for generating the recombinant constructs.

As a representative but non-limiting example, a useful expression vector for bacterial use comprises a selectable marker and bacterial origin of replication derived from a commercially available plasmid containing genetic elements of the well known cloning vector pBR322 (ATCC 37017). Such commercial vectors include, for example, pKK223-3 (Pharmacia Fine Chemicals, Uppsala, Sweden) and GEM 1 (Promega Biotech, Madison, Wis., USA). These pBR322 “backbone” sections are combined with an appropriate promoter and the structural sequence to be expressed. Other exemplary bacterial vectors include, for example, pBs, phagescript, PhiX174, pBluescript SK, pBs KS, pNH8a, pNH16a, pNH18a, pNH46a (Stratagene); pTrc99A, pKK223-3, pKK233-3, pDR540, and pRIT5. Preferably, vectors such as expression vectors will contain expression control sequence(s), e.g., promoters, that are regulatable in at least one host cell. Following transformation of a suitable host strain and growth of the host strain to an appropriate cell density, expression is induced or derepressed by appropriate means (e.g., temperature shift or chemical induction) and cells are cultured for an additional period.

Mammalian expression vectors comprise an origin of replication, a suitable promoter, and also any necessary ribosome binding sites, polyadenylation site, splice donor and acceptor sites, transcriptional termination sequences, and 5′ flanking nontranscribed sequences. DNA sequences derived from the SV40 viral genome, for example, the SV40 origin, early promoter, enhancer, splice, and polyadenylation sites may be used to provide the required expression control elements. Exemplary eukaryotic vectors include pcDNA3, pWLneo, pSV2cat, pOG44, PXTI, pSG (Stratagene) pSVK3, pBPV, pMSG, and pSVL.

Alternatively, a heterologous polynucleotide useful in the method of the invention may be operably linked to an expression control sequence such as the pMT2 or pED expression vectors disclosed in Kaufman et al., Nuc. Acids Res. 19:4485-4490, (1991), in order to produce the protein recombinantly. Many suitable expression control sequences are known in the art. General methods of expressing recombinant proteins are also known and are exemplified in R. Kaufman, Methods in Enzymology 185:537-566, (1990). As defined herein, “operably linked” means that two biomolecules, e.g., nucleotides, are joined or linked in a manner that preserved a capacity for interaction (e.g., a promoter and coding region interacting by proximity in the expression of the coding region.) For example, expression control sequences such as promoter regions can be selected from any desired gene. Bacterial promoters may include lacI, lacZ, T3, T7, gpt, lambda PR, and trc, and eukaryotic promoters include, for example, CMV immediate early, HSV thymidine kinase, early and late SV40, or LTR from retrovirus, and mouse metallothionein-I. Selection of an appropriate vector and promoter is well within the level of ordinary skill in the art.

The invention further provides host cells genetically engineered to contain the polynucleotides useful in the invention. Recombinant expression systems as defined herein will express polypeptides or proteins endogenous to the cell upon induction of a KIT receptor sequence element linked to endogenous DNA segment or gene to be expressed. The cells can be prokaryotic or eukaryotic. Host cells may contain nucleic acids of the invention introduced into the host cell using known transformation, transfection or infection methods.

Any host/vector system can be used to express one or more of the polynucleotides useful in the invention. Mature proteins can be expressed in mammalian cells, yeast, bacteria, or other cells under the control of appropriate promoters. Appropriate cloning and expression vectors for use with prokaryotic and eukaryotic hosts are described by Sambrook, et al., in Molecular Cloning: A Laboratory Manual, Second Edition, Cold Spring Harbor, N.Y. (1989), the disclosure of which is hereby incorporated by reference.

A number of types of cells may act as suitable host cell for expression of the protein. Mammalian host cells include, for example, monkey COS cells, Chinese Hamster Ovary (CHO) cells, human kidney HEK293 cells, human epidermal A431 cells, human Colo205 cells, 3T3 cells, CV-1 cells, other transformed primate cell lines, normal diploid cells, cell strains derived from in vitro culture of primary tissue, primary explants, HeLa cells, mouse L cells, BHK, HL-60, U937, HaK or Jurkat cells. Also contemplated for use as host cells for expressing the chimeric polypeptide are insect Sf9 cells. Any known viral expression system may also be used to generate the chimeric polypeptide, with adenovirus, retrovirus, baculovirus (as described in Summers et al., Texas Agricultural Experiment Station Bulletin No. 1555, 1987), and viral bacteriophages such as M13 or λ phage, being specifically contemplated.

C. Polypeptides for Use in the Method of the Invention

The isolated polypeptides useful in the method of the invention include, but are not limited to, a polypeptide comprising an amino acid sequence set forth as SEQ ID NO: 2 or an amino acid sequence encoded by the nucleotide sequence in SEQ ID NO: 1 or the corresponding full-length or mature protein. Polypeptides contemplated for use in the invention also include polypeptides retaining at least one biological or immunological activity of a KIT receptor, the polypeptides being encoded by any one of the following: (a) a polynucleotide having the nucleotide sequence set forth in SEQ ID NO: 1 or (b) polynucleotides encoding the amino acid sequences set forth as SEQ ID NO: 2 or (c) a polynucleotide that hybridizes to the complement of the polynucleotides of either (a) or (b) under stringent hybridization conditions. The invention also provides biologically active or immunologically active variants of any of the amino acid sequences set forth as SEQ ID NO: 2 or the corresponding full-length or mature protein; and variants thereof (e.g., with at least about 65%, at least about 70%, at least about 75%, at least about 80%, at least about 85%, 86%, 87%, 88%, 89%, at least about 90%, 91%, 92%, 93%, 94%, typically at least about 95%, 96%, 97%, more typically at least about 98%, or most typically at least about 99% amino acid identity) that retain biological activity. Polypeptides encoded by allelic variants may have a similar, increased, or decreased activity compared to a polypeptide comprising SEQ ID NO:2.

Fragments of the proteins contemplated by the present invention that are capable of exhibiting biological activity are also encompassed by the present invention. Fragments of the protein may be in linear form or they may be cyclized using known methods, for example, as described in Saragovi, et al., Bio/Technology 10:773-778, 1992 and in McDowell, et al., J. Amer. Chem. Soc. 114:9245-9253, 1992, each of which is incorporated herein by reference. Such a fragment may be fused to a carrier molecule such as an immunoglobulin for many purposes, including increasing the valency of a protein binding site.

The invention also provides for use of both full-length and mature forms of the disclosed proteins (for example, without a signal sequence or precursor sequence). The mature form of a protein is expected to be obtained by expression of a full-length polynucleotide in a homologous host cell. The sequence of the mature form of the protein is also determinable from the amino acid sequence of the full-length form. Where proteins contemplated by the present invention are membrane bound, soluble forms of the proteins are also provided. In such forms, part or all of the regions causing the proteins to be membrane-bound are deleted so that the proteins are fully secreted from the cell in which it is expressed. Protein compositions may further comprise an acceptable carrier, such as a hydrophilic, e.g., pharmaceutically acceptable, carrier.

The invention further provides isolated polypeptides encoded by the nucleic acid fragments of the present invention or by degenerate variants of the polynucleotide fragments of the present invention. By “degenerate variant” is intended nucleotide fragments which differ from a polynucleotide comprising a sequence expressly set forth herein (e.g., an open reading frame or ORF) by nucleotide sequence but, due to the degeneracy of the genetic code, encode an identical polypeptide sequence. Preferred polynucleotides of the present invention are the ORFs that encode proteins.

A variety of methodologies known in the art can be utilized to obtain any one of the isolated polypeptides or proteins contemplated for use in the present invention. For example, the amino acid sequence can be synthesized using commercially available peptide synthesizers. The synthetically constructed protein sequences, by virtue of sharing primary, secondary or tertiary structural and/or conformational characteristics with proteins are expected to possess biological properties in common therewith, including protein activity. This technique is particularly useful in producing small peptides and fragments of larger polypeptides. Fragments are useful, for example, in generating antibodies against the native polypeptide. Thus, they are useful as biologically active or immunological substitutes for natural, purified proteins in the screening of therapeutic compounds and in immunological processes for the development of antibodies.

The polypeptides and proteins useful in the invention can alternatively be purified from cells which have been altered to express the desired polypeptide or protein. As used herein, a cell is said to be altered to express a desired polypeptide or protein when the cell, through genetic manipulation, is made to produce a polypeptide or protein which it normally does not produce or which the cell normally produces at a lower level. One skilled in the art can readily adapt procedures for introducing and expressing either recombinant or synthetic sequences into eukaryotic or prokaryotic cells in order to generate a cell which produces one of the polypeptides or proteins useful in the method of the invention.

The invention also relates to methods for producing a polypeptide comprising growing a culture of host cells of the invention in a suitable culture medium, and purifying the protein from the cells or the culture in which the cells are grown. For example, the methods of the invention include a process for producing a polypeptide in which a host cell containing a suitable expression vector that includes a polynucleotide of the invention is cultured under conditions that allow expression of the encoded polypeptide. The polypeptide can be recovered from the culture, conveniently from the culture medium, or from a lysate prepared from the host cells and further purified. Preferred embodiments include those in which the protein produced by such process is a full length or mature form of the protein.

In an alternative method, the polypeptide or protein is purified from bacterial cells which naturally produce the polypeptide or protein. One skilled in the art can readily follow known-methods for isolating polypeptides and proteins in order to obtain one of the isolated polypeptides or proteins of the present invention. These includes but are not limited to, immunochromatography, HPLC, size-exclusion chromatography, ion-exchange chromatography, and immuno-affinity chromatography. See, e.g., Scopes, Protein Purification: Principles and Practice, Springer-Verlag (1994); Sambrook, et al., in Molecular Cloning: A Laboratory Manual; Ausubel et al., Current Protocols in Molecular Biology. Polypeptide fragments that retain biological/immunological activity include fragments comprising greater than about 100 amino acids, or greater than about 200 amino acids, and fragments that encode specific protein domains.

Modifications, in the peptide or DNA sequence, can be made by those skilled in the art using known techniques. Modifications of interest in the protein sequences may include the alteration, substitution, replacement, insertion or deletion of a selected amino acid residue in the coding sequence. For example, one or more of the cysteine residues may be deleted or replaced with another amino acid to alter the conformation of the molecule. Techniques for such alteration, substitution, replacement, insertion or deletion are well known to those skilled in the art (see, e.g., U.S. Pat. No. 4,518,584).

D. Screening Assays

Phosphotyrosine levels can be measured from transfected cells or cells isolated from biological samples by standard in vitro techniques well known in the art, such as enzyme-linked immunosorbant assay (ELISA), radio immunoassay (RIA), Western blot, or immunofluorescence-based assays. KIT activity can also be measured in biological samples using fluorescent microscopy with fluorescently labeled anti-KIT and anti-phosphotyrosine antibodies. The detection is correlated, for example, by a brighter staining signal in a fluorescent microscopy assay, the presence of more staining in a fluorescent microscopy assay, or by decreased fluid levels of phosphorylated KIT as detected by Western blot, ELISA, RIA or other immunofluorescence-based assays, such as fluorescence resonance electron transfer (FRET).

High-content screens (HCS) provide for analysis of multiple parameters in a single screening assay. For example, mutant or wild type KIT receptor activity may be measured using phosphotyrosine-specific antibodies fluorescing in a particular excitation channel (e.g., Alexa Fluor 488 excites at 488 nm). Antibodies to additional cell markers which excite at different wavelength ranges (i.e., in different channels) are then added to the same assay. High-content screens can analyze cell membrane proteins (e.g., activation, upregulation, or internalization) in response to KIT inhibitor to detect cell morphology, or actin/cytoskeletal proteins to detect cytoskeletal rearrangement, or nuclear staining to determine the extent of DNA replication or chromosome condensation and cell death. Immunohistochemical markers for mastocytosis that are useful in a high-content screen include trypase, CD34, CD68, KIT (CD117), and CD43 (Brockow et al., supra). It is further contemplated that the high-content screens of the invention can measure the downstream effects of a KIT modulator (e.g. inhibitor), i.e., any effects on proteins involved in normal receptor signaling pathways.

An anti-phosphotyrosine antibody suitable for use in the method of the invention may comprise a label, such as a radioisotope, a fluorophore, a fluorescing protein (e.g., natural or synthetic green fluorescent proteins), a dye, an enzyme, a substrate, or the like. The label is a compound or moiety known in the art to be useful as a label, including biotin molecules, alkaline phosphatase, fluorophores (e.g., fluoroisothiocyanate, phycoerythrin, Texas red, Alexa Fluor stains, and other fluorescent dyes well known in the art), radioisotopes (e.g., ³H, Europium³⁺, ³²P), genetically engineered peptide tags such as a histidine (His₆) tag linked to the aggregating polypeptide, a myc-tag, a Hemagluttinin tag, and the like. Biotin, fluorophores, and other contemplated small molecules comprising a label can be linked to the polypeptide of the invention by means well-known in the art such as a commercially produced Biotinylation kit (Sigma Chem. Co., St. Louis, Mo.), or alternative methods commonly used in organic chemistry to attach a small molecule to a peptide or protein (see e.g., Current Protocols in Protein Chemistry, John Wiley & Sons, 2001). Genetically engineered tags, e.g., His₆ and myc-tags, are operably linked to the polypeptide of the invention using standard recombinant DNA methods well known in the art (see e.g., Current Protocols in Molecular Biology, supra), or using conventional peptide synthesis techniques. Such labels facilitate quantitative detection with standard laboratory machinery and techniques.

The candidate inhibitor employed in the method of the invention can be any organic or inorganic chemical or biological molecule known in the art, such as small organic or inorganic molecules preferably found in small molecule libraries containing compounds of synthetic or natural origin, or combinatorial libraries as described below. Further, peptides, preferably found in peptide libraries, are contemplated as candidate modulators such as inhibitors. Preferred candidate modulators (e.g., inhibitors) are suitable for administration as therapeutics and will, therefore, preferably exhibit acceptable toxicity levels as would be known in the art or determinable by one of skill in the art using routine experimentation. Toxicity can be determined in subsequent assays, however, and often “designed out” of molecules by pharmaceutical chemists. Screening of chemical libraries such as those developed and maintained by pharmaceutical companies, consisting of both chemically synthesized and natural compounds, and combinatorial libraries, is specifically contemplated.

Chemical libraries may contain known compounds, proprietary structural analogs of known compounds, or compounds that are identified from natural product screening.

Natural product libraries are collections of materials isolated from natural sources, typically, microorganisms, animals, plants, or marine organisms. Natural products are isolated from their sources by fermentation of microorganisms followed by isolation and extraction of the fermentation broths or by direct extraction from the microorganism or tissue (plant or animal) themselves. Natural product libraries include polyketides, non-ribosomal peptides, and variants (including non-naturally occurring variants) thereof. See Cane et al., Science, 282:63-68 (1998), incorporated herein by reference.

Combinatorial libraries are composed of large numbers of related compounds, such as peptides, oligonucleotides, or other organic compounds as a mixture. Such compounds are relatively straightforward to design and prepare by traditional automated synthesis protocols, PCR, cloning or proprietary synthetic methods. Of particular interest are peptide and oligonucleotide combinatorial libraries.

Still other libraries of interest include peptide, protein, peptidomimetic, multiparallel synthetic collection, recombinatorial, and polypeptide libraries. For a review of combinatorial chemistry and libraries created thereby, see Myers, Curr. Opin. Biotechnol., 8:701-707 (1997), incorporated herein by reference.

Inhibitors identified by assessment of the candidate modulators (e.g., inhibitors) may be formulated into compositions which include pharmaceutically acceptable (i.e., sterile and non-toxic) liquid, semisolid, or solid diluents that serve as pharmaceutical vehicles, excipients, or media. Inhibitors formulated in this manner can be further screened for modulating activity in vivo, e.g. in animal models for disease, or can be administered to humans in clinical trials, or can be made and sold as pharmaceuticals. Modulator compositions according to the invention may be administered in any suitable manner using an appropriate pharmaceutically acceptable vehicle, e.g., a pharmaceutically acceptable diluent, adjuvant, excipient or carrier. The composition preferably comprises a pharmaceutically acceptable carrier solution such as water, saline, phosphate-buffered saline, glucose, or other carriers conventionally used to deliver therapeutics.

The inhibitor compositions can be packaged in forms convenient for delivery. The compositions can be enclosed within a capsule, caplet, sachet, cachet, gelatin, paper, or other container. The dosage units can be packaged, e.g. in tablets, capsules, suppositories or cachets.

E. Antibodies

Antibodies useful for detecting peptides comprising phosphorylated tyrosine are generated using techniques well known in the art. Thus, the invention contemplates use of antibodies (e.g., monoclonal and polyclonal antibodies, single chain antibodies, chimeric antibodies, bifunctional/bispecific antibodies, humanized antibodies, human antibodies, and complementarity determining region (CDR)-grafted antibodies, including compounds that include CDR sequences specifically recognizing a polypeptide of the invention and specific for polypeptides of interest to the invention, especially phosphorylated tyrosine on the KIT receptor). Preferred antibodies are human antibodies which are produced and identified according to methods described in WO 93/11236, which is incorporated herein by reference in its entirety. Antibody fragments, including Fab, Fab′, F(ab′)₂, and Fv, and single-chain antibodies are also provided by the invention. The term “specific for,” when used to describe antibodies of the invention, indicates that the variable regions of the antibodies of the invention recognize and bind the polypeptide of interest with a detectable preference (i.e., able to distinguish the polypeptide of interest from other known polypeptides of the same family, by virtue of measurable differences in binding affinity, despite the possible existence of localized sequence identity, homology, or similarity between family members). It will be understood that specific antibodies may also interact with other proteins (for example, S. aureus protein A or other antibodies in ELISA techniques) through interactions with sequences outside the variable region of the antibodies, and in particular, in the constant region of the molecule. Screening assays to determine binding specificity of an antibody of the invention are well known and routinely practiced in the art. For a comprehensive discussion of such assays, see Harlow et al. (Eds), Antibodies A Laboratory Manual; Cold Spring Harbor Laboratory; Cold Spring Harbor, N.Y. (1988), Chapter 6. Antibodies of the invention can be produced using any method well known in the art.

Various procedures known in the art may be used for the production of polyclonal antibodies to peptides comprising phosphorylated tyrosine. For the production of antibodies, various host animals (including but not limited to rabbits, mice, rats, hamsters, and the like) are immunized by injection with a phosphorylated KIT protein or peptide. Various adjuvants may be used to increase the immunological response, depending on the host species; including but not limited to Freund's (complete and incomplete) adjuvant, mineral gels such as aluminium hydroxide, surface active substances such as lysolecithin, pluronic polyols, polyanions, oil emulsions, keyhole limpet hemocyanins, dinitrophenol, and potentially useful human adjuvants such as BCG (Bacillus Calmette-Guerin) and Corynebacterium parvunt.

A monoclonal antibody to a phosphorylated epitope of KIT may be prepared by using any technique which provides for the production of antibody molecules by continuous cell lines in culture. These include but are not limited to the hybridoma technique originally described by Köhler et al., Nature, 256: 495-497 (1975), and the more recent human B-cell hybridoma technique [Kosbor et al., Immunology Today, 4: 72 (1983)] and the EBV-hybridoma technique [Cole et al., Monoclonal Antibodies and Cancer Therapy, Alan R Liss, Inc., pp. 77-96 (1985), all specifically incorporated herein by reference]. Antibodies against phosphorylated KIT also may be produced in bacteria from cloned immunoglobulin cDNAs. With the use of the recombinant phage antibody system it may be possible to quickly produce and select antibodies in bacterial cultures and to genetically manipulate their structure.

When the hybridoma technique is employed, myeloma cell lines may be used. Such cell lines suited for use in hybridoma-producing fusion procedures preferably are non-antibody-producing, have high fusion efficiency, and exhibit enzyme deficiencies that render them incapable of growing in certain selective media which support the growth of only the desired fused cells (hybridomas). For example, where the immunized animal is a mouse, one may use P3-X63/Ag8, P3-X63-Ag8.653, NS1/1.Ag 4 1, Sp210-Ag14, FO, NSO/U, MPC-11, MPC11-X45-GTG 1.7 and S194/5XX0Bul; for rats, one may use R210.RCY3, Y3-Ag 1.2.3, IR983F and 4B210; and U-266, GM1500-GRG2, LICR-LON-HMy2 and UC729-6 are all useful in connection with cell fusions. It should be noted that the hybridomas and cell lines produced by such techniques for producing the monoclonal antibodies are contemplated compositions of the present invention.

In addition to the production of monoclonal antibodies, techniques developed for the production of “chimeric antibodies,” the splicing of mouse antibody genes to human antibody genes to obtain a molecule with appropriate antigen specificity and biological activity can be used [Morrison et al., Proc. Natl. Acad. Sci. 81:6851-6855 (1984); Neuberger et al., Nature 312:604-608 (1984); Takeda et al., Nature 314:452-454 (1985)]. Alternatively, techniques described for the production of single-chain antibodies (U.S. Pat. No. 4,946,778) can be adapted to produce phosphorylated-KIT peptide-specific single chain antibodies.

Antibody fragments which contain the idiotype of the molecule may be generated by known techniques. For example, such fragments include, but are not limited to, the F(ab′)₂ fragment which may be produced by pepsin digestion of the antibody molecule; the Fab′ fragments which may be generated by reducing the disulfide bridges of the F(ab′)₂ fragment, and the two Fab fragments which may be generated by treating the antibody molecule with papain and a reducing agent.

Non-human antibodies may be humanized by any methods known in the art. A preferred “humanized antibody” has a human constant region, while the variable region, or at least a CDR, of the antibody is derived from a non-human species. Methods for humanizing non-human antibodies are well known in the art. (see U.S. Pat. Nos. 5,585,089, and 5,693,762). Generally, a humanized antibody has one or more amino acid residues introduced into its framework region from a source which is non-human. Humanization can be performed, for example, using methods described in Jones et al., Nature 321: 522-525, (1986), Riechmann et al., Nature, 332: 323-327, (1988) and Verhoeyen et al., Science 239:1534-1536, (1988), by substituting at least a portion of a rodent complementarity-determining region for the corresponding regions of a human antibody. Numerous techniques for preparing engineered antibodies are described, e.g., in Owens et al., J. Immunol. Meth., 168:149-165, (1994). Further changes can then be introduced into the antibody framework to modulate affinity or immunogenicity.

Rapid, large-scale recombinant methods for generating antibodies may be employed, such as phage display [Hoogenboom et al., J. Mol. Biol. 227: 381, (1991); Marks et al, J. Mol. Biol. 222: 581, (1991)] or ribosome display methods, optionally followed by affinity maturation [see, e.g., Ouwehand et al., Vox Sang 74(Suppl 2):223-232 (1998); Rader et al., Proc. Natl. Acad. Sci. USA 95:8910-8915 (1998); Dall'Acqua et al., Curr. Opin. Struct. Biol. 8:443-450, (1998)]. Phage-display processes mimic immune selection through the display of antibody repertoires on the surface of filamentous bacteriophage, and subsequent selection of phage by their binding to an antigen of choice. One such technique is described in WO 99/10494, which describes the isolation of high affinity and functional agonistic antibodies for MPL and msk receptors using such an approach.

Bispecific antibodies are monoclonal, preferably human or humanized, antibodies that have binding specificities for at least two different antigens. Bispecific antibodies are produced, isolated, and tested using standard procedures that have been described in the literature. See, e.g., Pluckthun et al., Immunotechnology, 3:83-105 (1997); Carter et al., J. Hematotherapy, 4: 463-470 (1995); Renner & Pfreundschuh, Immunological Reviews, 1995, No. 145, pp. 179-209; Pfreundschuh U.S. Pat. No. 5,643,759; Segal et al., J. Hematotherapy, 4: 377-382 (1995); Segal et al., Immunobiology, 185: 390-402 (1992); and Bolhuis et al., Cancer Immunol. Immunother., 34: 1-8 (1991), all of which are incorporated herein by reference in their entireties.

The term “bispecific antibody” refers to a single, bivalent antibody which has two different antigen binding sites (variable regions). As described below, the bispecific binding agents are generally made of antibodies, antibody fragments, or analogs of antibodies containing at least one complementarity determining region derived from an antibody variable region. These may be conventional bispecific antibodies, which can be manufactured in a variety of ways (Holliger et al., Current Opinion Biotechnol. 4, 446-449 (1993)), e.g., prepared chemically, using hybrid hybridomas, by placing the coding sequence of such a bispecific antibody into a vector and producing the recombinant peptide, or by phage display. The bispecific antibodies may also be any bispecific antibody fragments.

In one method, bispecific antibodies fragments are constructed by converting whole antibodies into (monospecific) F(ab′)₂ molecules by proteolysis, splitting these fragments into the Fab′ molecules and recombine Fab′ molecules with different specificity to bispecific F(ab′)₂ molecules (see, for example, U.S. Pat. No. 5,798,229).

A bispecific antibody can be generated by enzymatic conversion of two different monoclonal antibodies, each comprising two identical L (light chain)-H (heavy chain) half molecules and linked by one or more disulfide bonds. Each monoclonal antibody is converted into two F(ab′)₂ molecules, splitting each F(ab′)₂ molecule under reducing conditions into the Fab′ thiols. One of the Fab′ molecules of each antibody is activated with a thiol activating agent and the active Fab′ molecule are combined, wherein an activated Fab′ molecule bearing one specificity is linked with a non-activated Fab′ molecule bearing an second specificity or vice versa in order to obtain the desired bispecific antibody F(ab′)₂ fragment.

Another method for producing bispecific antibodies is by the fusion of two hybridomas to form a hybrid hybridoma, as defined previously. Using now standard techniques, two antibody producing hybridomas are fused to give daughter cells, and those cells that have maintained the expression of both sets of clonotype immunoglobulin genes are then selected.

To identify the bispecific antibody, standard methods such as ELISA are used wherein the wells of microtiter plates are coated with a reagent that specifically interacts with one of the parent hybridoma antibodies and that lacks cross-reactivity with both antibodies. In addition, FACS, immunofluorescence staining, idiotype specific antibodies, antigen binding competition assays, and other methods common in the art of antibody characterization may be used in conjunction with the present invention to identify preferred hybrid hybridomas.

Recombinant antibody fragments, e.g., scFvs, can also be engineered to assemble into stable multimeric oligomers of high binding avidity and specificity to different target antigens. Such diabodies (dimers), triabodies (trimers) or tetrabodies (tetramers) are well known in the art, see e.g., Kortt et al., Biomol Eng. 2001 18:95-108, (2001) and Todorovska et al., J Immunol Methods. 248:47-66, (2001).

F. Formulation of Pharmaceutical Compounds

It is contemplated that candidate inhibitors identified by the method of the invention as KIT inhibitors are administered to a subject in composition with one or more pharmaceutically acceptable carriers. It is further contemplated that candidate inhibitors identified by the invention as KIT inhibitors are formulated in a pharmaceutical composition with one or more chemotherapeutic agents, such as an anti-metabolites, a DNA-damaging agent, a cytokine, a covalent DNA-binding drug, a topoisomerase inhibitor, an anti-tumor antibiotic, a differentiation agent, an alkylating agent, a methylating agent, a nitrogen mustard, or other therapeutic agents, as identified above or known in the art.

Pharmaceutical carriers used in the invention include pharmaceutically acceptable salts, particularly where a basic or acidic group is present in a compound. For example, when an acidic substituent, such as —COOH, is present, the ammonium, sodium, potassium, calcium and the like salts, are contemplated as preferred embodiments for administration to a biological host. When a basic group (such as amino or a basic heteroaryl radical, such as pyridyl) is present, then an acidic salt, such as hydrochloride, hydrobromide, acetate, maleate, pamoate, phosphate, methanesulfonate, p-toluenesulfonate, and the like, is contemplated as a preferred form for administration to a biological host.

Similarly, where an acid group is present, then pharmaceutically acceptable esters of the compound (e.g., methyl, tert-butyl, pivaloyloxymethyl, succinyl, and the like) are contemplated as preferred forms of the compounds, such esters being known in the art for modifying solubility and/or hydrolysis characteristics for use as sustained release or prodrug formulations. In addition, some compounds may form solvates with water or common organic solvents. Such solvates are contemplated as well.

Pharmaceutical inhibitor compositions can be used directly to practice materials and methods of the invention, but in preferred embodiments, the compounds are formulated with pharmaceutically acceptable diluents, adjuvants, excipients, or carriers. The phrase “pharmaceutically or pharmacologically acceptable” refer to molecular entities and compositions that do not produce adverse, allergic, or other untoward reactions when administered to an animal or a human, e.g., orally, topically, transdermally, parenterally, by inhalation spray, vaginally, rectally, or by intracranial injection. (The term parenteral as used herein includes subcutaneous injections, intravenous, intramuscular, intracisternal injection, or infusion techniques. Administration by intravenous, intradermal, intramusclar, intramammary, intraperitoneal, intrathecal, retrobulbar, intrapulmonary injection and or surgical implantation at a particular site is contemplated as well.) Generally, this will also entail preparing compositions that are essentially free of pyrogens, as well as other impurities that could be harmful to humans or animals. The term “pharmaceutically acceptable carrier” includes any and all solvents, dispersion media, coatings, antibacterial and antifungal agents, isotonic and absorption delaying agents and the like. The use of such media and agents for pharmaceutically active substances is well known in the art.

The pharmaceutical compositions containing the KIT inhibitors described above may be in a form suitable for oral use, for example, as tablets, troches, lozenges, aqueous or oily suspensions, dispersible powders or granules, emulsions, hard or soft capsules, or syrups or elixirs. Compositions intended for oral use may be prepared according to any known method, and such compositions may contain one or more agents selected from the group consisting of sweetening agents, flavoring agents, coloring agents and preserving agents in order to provide pharmaceutically elegant and palatable preparations. Tablets may contain the active ingredient in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia; and lubricating agents, for example magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed. They may also be coated by the techniques described in the U.S. Pat. Nos. 4,256,108; 4,166,452; and 4,265,874 to form osmotic therapeutic tablets for controlled release.

Formulations for oral use may also be presented as hard gelatin capsules wherein the active ingredient is mixed with an inert solid diluent, for example, calcium carbonate, calcium phosphate or kaolin, or as soft gelating capsules wherein the active ingredient is mixed with water or an oil medium, for example peanut oil, liquid paraffin, or olive oil.

Aqueous suspensions may contain the active compounds in admixture with excipients suitable for the manufacture of aqueous suspensions. Such excipients are suspending agents, for example sodium carboxymethylcellulose, methylcellulose, hydroxypropylmethylcellulose, sodium alginate, polyvinylpyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl, p-hydroxybenzoate, one or more coloring agents, one or more flavoring agents, and one or more sweetening agents, such as sucrose or saccharin.

Oily suspensions may be formulated by suspending the active ingredient in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as ascorbic acid.

Dispersible powders and granules suitable for preparation of an aqueous suspension by the addition of water provide the active compound in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

The pharmaceutical compositions of the invention may also be in the form of oil-in-water emulsions. The oily phase may be a vegetable oil, for example olive oil or arachis oil, or a mineral oil, for example liquid paraffin or mixtures of these. Suitable emulsifying agents may be naturally-occurring gums, for example gum acacia or gum tragacanth, naturally-occurring phosphatides, for example soy bean, lecithin, and esters or partial esters derived from fatty acids and hexitol anhydrides, for example sorbitan monoleate, and condensation products of the said partial esters with ethylene oxide, for example polyoxyethylene sorbitan monooleate. The emulsions may also contain sweetening and flavoring agents.

Syrups and elixirs may be formulated with sweetening agents, for example glycerol, propylene glycol, sorbitol or sucrose. Such formulations may also contain a demulcent, a preservative and flavoring and coloring agents. The pharmaceutical compositions may be in the form of a sterile injectable aqueous or oleaginous suspension. This suspension may be formulated according to the known art using those suitable dispersing or wetting agents and suspending agents which have been mentioned above. The sterile injectable preparation may also be a sterile injectable solution or suspension in a non-toxic parenterally-acceptable diluent or solvent, for example as a solution in 1,3-butane diol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

The compositions may also be in the form of suppositories for rectal administration of the PTPase modulating compound. These compositions can be prepared by mixing the drug with a suitable non-irritating excipient which is solid at ordinary temperatures but liquid at the rectal temperature and will therefore melt in the rectum to release the drug. Such materials are cocoa butter and polyethylene glycols, for example.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial an antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

G. Administration and Dosing

Some methods of the invention include a step of polypeptide administration to a human or animal. Polypeptides may be administered in any suitable manner using an appropriate pharmaceutically-acceptable vehicle, e.g., a pharmaceutically-acceptable diluent, adjuvant, excipient or carrier. The composition to be administered according to methods of the invention preferably comprises a pharmaceutically-acceptable carrier solution such as water, saline, phosphate-buffered saline, glucose, or other carriers conventionally used to deliver therapeutics or imaging agents.

The “administering” that is performed according to the present invention may be performed using any medically-accepted means for introducing a therapeutic directly or indirectly into a mammalian subject, including but not limited to injections (e.g., intravenous, intramuscular, subcutaneous, intracranial or catheter); oral ingestion, intranasal or topical administration; and the like. In one embodiment, administering the composition is performed at the site of a lesion or affected tissue needing treatment by direct injection into the lesion site or via a sustained delivery or sustained release mechanism, which can deliver the formulation internally. For example, biodegradable microspheres or capsules or other biodegradable polymer configurations capable of sustained delivery of a composition (e.g., a soluble polypeptide, antibody, or small molecule) can be included in the formulations of the invention implanted near the lesion.

The therapeutic composition may be delivered to the patient at multiple sites. The multiple administrations may be rendered simultaneously or may be administered over a period of several hours. In certain cases it may be beneficial to provide a continuous flow of the therapeutic composition. Additional therapy may be administered on a period basis, for example, daily, weekly or monthly.

Polypeptides or inhibitors for administration may be formulated with uptake or absorption enhancers to increase their efficacy. Such enhancers include for example, salicylate, glycocholate/linoleate, glycholate, aprotinin, bacitracin, SDS caprate and the like. See, e.g., Fix (J. Pharm. Sci., 85:1282-1285, 1996) and Oliyai and Stella (Ann. Rev. Pharmacol. Toxicol., 32:521-544, 1993).

The amounts of pharmaceutical composition in a given dosage will vary according to the size of the individual to whom the therapy is being administered as well as the characteristics of the disorder being treated. In exemplary treatments, it may be necessary to administer about 50 mg/day, 75 mg/day, 100 mg/day, 150 mg/day, 200 mg/day, 250 mg/day, 500 mg/day or 1000 mg/day. These concentrations may be administered as a single dosage form or as multiple doses. Standard dose-response studies, first in animal models and then in clinical testing, reveal optimal dosages for particular disease states and patient populations.

It will also be apparent that dosing should be modified if traditional therapeutics are administered in combination with inhibitors identified by the invention. For example, treatment of mast cell disorders and related leukemias or other cancers using traditional chemotherapeutics or radiotherapeutics, in combination with inhibitors identified by the invention, is contemplated. In some embodiments, KIT inhibitors identified by the invention are administered to patients in combination with chemotherapeutic agents wherein the compositions are administered simultaneously. In other embodiments, KIT inhibitors identified by the invention may be administered to a patient either before treatment with chemo- or radiotherapeutic agents or after treatment with chemo- or radiotherapeutic agents. The inhibitors identified by the invention may be administered in combination with one or more chemotherapeutic agents up to two weeks before, up to one week before, up to one day before, or up to one hour before treatment with one or more chemotherapeutic agents. In still other embodiments, the inhibitors identified by the invention may be administered in combination with one or more chemotherapeutic agents up to two weeks after, up to one week after, up to one day after, or up to one hour after treatment with chemotherapeutic agents. The particular treatment regimen is determined by those of skill in the art on a case-by-case basis, using no more than routine experimentation and optimization techniques to determine an appropriate course of treatment in each case.

H. Kits

As an additional aspect, the invention includes kits which comprise one or more compounds or compositions of the invention packaged in a manner which facilitates their use to practice methods of the invention. In a simplest embodiment, such a kit includes a compound or composition described herein as useful for practice of a method of the invention (e.g., inhibitors of active KIT receptor and phosphotyrosine antibody for use in screening assays), packaged in a container such as a sealed bottle or vessel, with a label affixed to the container or included in the package that describes use of the compound or composition to practice the method of the invention. Preferably, the compound or composition is packaged in a unit dosage form. The kit may further include a device suitable for administering the composition according to a preferred route of administration or for practicing a screening assay.

Additional aspects and details of the invention will be apparent from the following examples, which are intended to be illustrative rather than limiting.

### EXAMPLE 1

### Recombinant KITD816V Expressed from HEK93 Cells is Highly Active In Vivo

Mutated variants of the KIT tyrosine kinase receptor that play a significant role in the development of mastocytosis, and other disorders associated with aberrant mast cell proliferation, exhibit high levels of auto-phosphorylation. Typically, cell-based high-throughput screen (HTS) kinase assays are performed in order to study the effects of potential inhibitor compounds oil the receptor phosphorylation state. These HTSs require large amounts of purified protein to accurately carry out phosphorylation analysis.

A barrier to expression of mutant KIT receptor in standard recombinant protein systems, such as bacteria or yeast cell lines, is the toxicity of the mutant KIT receptor, perhaps attributed to the high degree of tyrosine kinase activity. For example, the KITD814V activated mutant expressed in bacterial recombinant systems or yeast Pichia pastoris are difficult to purify in adequate amounts due to toxicity. Additionally, any receptor purified from these bacterial or yeast systems expresses a KIT receptor with low activity.

To enable the study of KITD816V mutant activity, a recombinant method was developed to overcome the aforementioned difficulties. To establish a recombinant system effectively producing KIT mutants, human embryonic kidney HEK293 cells were transiently transfected with a plasmid containing the KITD816V mutant receptor.

HEK cells were maintained at 37° C. and 5% CO₂ in modified Eagle's medium supplemented with 10% fetal bovine serum, 2 mM L-glutamine, 1 mM pyruvate, 0.1 mM non-essential amino acids (GIBCO BRL) 50 U/ml penicillin, and 50 μg/ml streptomycin. HEK cells expressing different KIT mutants were maintained in the above medium supplemented with 0.4 mg/ml geneticin for clone selection purposes. Transient transfection of HEK293 cells was performed using calcium phosphate co-precipitation of DNA. To establish stable cell lines, HEK293 cells were transfected with the pcDNA3.1 expression vector containing polynucleotide encoding either wild-type KIT (KITWT), KITD816V mutant receptor, KITΔK550-558 deletion mutant, or KITY823F, wherein the tyrosine phosphorylation site has been replaced with non-phosphorylatable phenylalanine residue. Two days after transfection, geneticin was added to the culture media and geneticin-resistant cells were pooled. Single clones were established by limiting dilution assay.

Subsequent analysis of the isolated clonal cell lines were directed towards characterization of the expressed KIT mutants, described here in terms of the HEK clone expressing KITD816V. The cells were lysed and total lysate analyzed to determine the activation state of the receptor. SDS-PAGE and immunoblot revealed that recombinant mutant receptor expressed in HEK cells demonstrates high levels of tyrosine phosphorylation independent of ligand induction.

### EXAMPLE 2

### Production of Antibodies to Phosphorylated KIT Receptor

In order to accurately measure the phosphorylation state of a constitutively active KIT mutant, an antibody that specifically recognizes KIT activated by auto-phosphorylation rather than KIT phosphorylation, e.g., resulting from a intracellular protein-protein-interaction, was elicited. Tyrosine 823 in the activation loop of the KIT receptor is the primary site of auto-phosphorylation in activated Kit and is a good target for detecting constitutively active KIT mutants.

Polyclonal antiserum that recognizes a phosphorylated tyrosine 823 was elicited to a phosphopeptide corresponding to 11 amino acids in the KIT receptor, KNDSNY₈₂₃VVKGN, containing the phosphotyrosine site (residues 818-828 of SEQ ID NO:2). The peptide was synthesized using conventional phospho-peptide synthesis technology (Affinity Bioreagents, Golden, Colo.), according to the manufacturer's protocol.

The synthetic polypeptide was coupled to a carrier protein and injected into rabbits using standard immunization techniques performed by Affinity Bioreagents, Inc. (Golden, Colo.). Rabbit polyclonal antibody specific for pY823-Kit (pY823) was purified using epitope affinity chromatography (Affinity Bioreagents, Inc.).

To assess the specificity of pY823 antibodies, serum-starved HEK293 cells transiently transfected with KITWT, KITY823F or KITD816V expression vectors were treated with stem cell factor (150 ng/ml) (Calbiochem, San Diego, Calif.) for 7 minutes at 37° C. and lysed in lysis buffer (1% Triton X-100, 20 mM Tris HCl (pH7.4), 80 mM NaCl, 5 mM EDTA, 10% glycerol, 1 mM phenylmethylsulfonyl fluoride (PMSF), 10 μg/ml leupeptin, 10 μg/ml aprotinin, 30 mM NA₄P₂O₇, 250 μM Na₃V₄, 50 mM NF, 40 μM phenylarsine oxide). Cell extracts were precleared by centrifugation and analyzed by gel electrophoresis (SDS-PAGE) or incubated with antibodies cross-linked to protein A-SEPHAROSE™ beads in a nutator at 4° C. for 2 hours.

Lysates were immunoprecipitated with either Kit-C1 antibody, specific for the C-terminus of Kit receptor (Blume-Jensen et al., EMBO J. 12:4199-4209. 1993), pY823 antibody, or pY823 antibody neutralized with immunizing peptide. Immunoprecipitated material was separated on SDS-PAGE, transferred to nitrocellulose filters, and exposed to either the pY823 or the Kit-C1 antiserum. The same blot was re-probed with pY99 antibody, a monoclonal antibody specific for phosphotyrosine (Transduction Laboratories, San Jose, Calif.) as a control for SCF activation, and also with Kit-C1 and pY823 antisera to control for protein expression levels.

Immunoblot analysis of transfected HEK293 cells demonstrated that pY823 antibody recognized KITWT immunoprecipitated either with Kit-C1 or pY823, but only from SCF-stimulated cells, indicating that the pY823 antibody specifically recognizes only activated KIT. Immunoprecipitation in the presence of the neutralizing peptide completely blocked detection of the stimulated receptor. The pY823 antibody failed to recognize the tyrosine to phenylalanine mutant (Y823F). In contrast, the pY823 antibody recognized the constitutively active D816V mutant with high affinity.

To assess the pY823 antibody in immunofluorescence assays, HEK293 cells were grown on poly-L-lysine coated cover slips (Becton Dickinson, Mountain View, Calif.) and transfected with the KITWT expression vector. The cells were then serum starved and either treated with SCF as above or left untreated. Cells were washed in phosphate-buffered saline (PBS), fixed in 4% paraformaldehyde for 20 minutes at room temperature, permeabilized for 20 minutes in PBS with 0.2% Triton X-100, and washed. Coverslips were stained 1 hour with the KIT specific antibody A4502 (Dako, Inc., Carpinteria, Calif.) to assess transfection efficiency, with pY823 or with control serum. Cells were then washed and stained (1 hour) with AlexaFluor 488 conjugated donkey anti-rabbit IgG specific secondary antibody (Molecular Probes, Inc., Eugene, Oreg.), washed in PBS, and mounted in Vectashield (Vector Laboratories, Inc., Burlingame, Calif.) for analysis by fluorescence microscopy.

The pY823 antibody detected phosphorylated KITWT only in SCF-stimulated cells, while the antibody strongly recognized tyrosine phosphorylation in KITD816V in transiently transfected HEK293 cells in an SCF-independent manner. Phosphorylation was completely abolished in KITD816V transfected cells pretreated with the kinase inhibitor, staurosporin (10 μM) (Calbiochem) and partially decreased in cells treated with 1 μM staurosporin.

Cells were then assayed in the presence of the FDA approved c-Kit inhibitor Gleevec (Glivac, STI571), which has been shown to inhibit KIT phosphorylation in gastrointestinal stromal tumors (GISTs) expressing mutant KIT. Previous studies demonstrated that Gleevec is ineffective against KIT816 mutants (Heinrich et al., J. Clin. Oncol. 20:1692-1703, 2002; Zermati et al., Oncogene 22:660-4, 2003). HEK cells transiently transfected to express KITD816V and pretreated with Gleevec gave a strong signal in the presence of pY823, indicating that Gleevec did not inhibit KIT autophosphorylation in transiently transfected KITD816V-expressing HEK cells

The result indicates that the mutant KIT was constitutively active in HEK cells, was not highly toxic to the cells, and also that the present method of screening inhibitors is considerably more sensitive than previous in vitro phosphorylation assays of mutant KIT receptors. These in vitro studies, however, were not sensitive enough to detect STI571 (Gleevec) inhibition of D816V, possibly due to inadequate KIT protein amounts or the loss of phosphorylation during the purification process.

Based on the above results, it is expected that stable cell lines expressing KIT mutants will be useful tools for assessing the phosphorylation state of KIT and for detecting modulators (e.g., inhibitors) of KIT activation.

### EXAMPLE 3

### HEK293 Cells Stably Expressing KIT Mutants Demonstrate High Levels of Auto-Phosphorylation

To analyze the activity of KIT mutants and to identify modulators of KIT mutants, stable cells lines were created that express any of a variety of KIT receptors, including KITWT, as well as the KIT mutants KITD816V and KITΔK550-558, and the control mutant KITY823F. It is expected that stable cells lines expressing any clinically relevant KIT receptor mutation, such as D816H, D816F, D816Y, or D816N, may be generated using the methods described herein.

Stable cell lines derived from HEK293 cells and containing an exogenous KIT mutant coding region were generated as described in Example 1. Stable cell lines were generated that expressed either wild type Kit (KITWT), KITD816V mutant, KITΔK550-558 deletion mutant, or KITY823F. Single clones were picked and analyzed for KIT expression by immunofluorescent staining with anti-KIT A4502 antibodies as described previously. Clones showing stable homogeneous expression of KITWT and mutant KIT were selected for further analyses.

Transfected HEK cells were serum-starved and subsequently treated with SCF as above (or untreated as a control), lysed, and either total lysate or KIT-C1 immunoprecipitated lysate was subjected to SDS-PAGE, controlling for total protein content to ensure that total protein levels in each sample were equivalent. Protein was transferred to nitrocellulose membranes and the blots were analyzed, as described above, with pY823 antibody and control antibodies (Kit-C1).

Immunoprecipitated lysate and total lysate showed comparable protein expression for each of KITWT, KITΔK550-558, and KITY823F, while KITD816V was expressed at lower levels, perhaps due to its toxicity to cells. However, pY823 demonstrated strong recognition of KITD816V in an SCF-independent manner. KITΔK550-558 exhibited background levels of phosphorylation, as detected with pY823 and pY99 antibodies, while KITY823F was not recognized by either pY823 or pY99.

The cell-based assay for screening candidate inhibitors of constitutively activated KIT receptor is, therefore, highly sensitive compared to in vitro, test tube based assays. This cell-based expression method avoids the primary drawback of test tube assays, which is the need for substantial amounts of purified protein. The potential toxicity of highly phosphorylated mutant KIT is rendered less significant using a cell-based assay comprising mammalian cells that stably express the KIT mutants. The increased detection sensitivity of the cell-based assay allows for measurement of KIT receptor activation at considerably lower levels of the protein that previously possible. Additionally, this cell-based method provides a quantitative method for assessing KIT inhibition in a variety of formats, including high-throughput assay, high-content screening formats.

### EXAMPLE 4

### High-Throughput Assay Useful in Screening for Inhibitors of Activated KIT

As noted above, the in vitro assays typically used to detect tyrosine phosphorylation have proven problematic is assessing mutant KIT activation due to the inability to purify sufficient amounts of the relevant mutant KIT protein. To analyze the ability of compounds to inhibit activated KIT receptors, either SCF-stimulated wild-type receptor or constitutively active KIT mutant, a high-throughput assay utilizing immunofluorescence detection of phosphorylated KIT was developed.

A KITD816V-expressing HEK293 cell line was seeded in triplicate in 384-well poly-D-lysine-coated black clear bottom plates (Becton-Dickinson) in 85 μL volume and grown at 37° C./5% CO₂. After 24 hours, KIT inhibitors or DMSO prediluted in growth medium were added to a final volume of 90 μL/well for 30 minutes. Media was removed using a multichannel aspirator, and the cells were fixed in prewarmed (37° C.) 4% paraformaldehyde for 20 minutes at room temperature. Cells were permeabilized in PBS/0.2% Triton X-100 for 20 minutes and washed 2× in PBS. Cells were exposed to pY823 or control antibody and detected with conjugated anti-rabbit IgG as described above. Total immunofluorescence was measured using ANALYST™. Plates were also analyzed using The DISCOVERY-1™ High-Content Screening System from Molecular Devices (Downingtown, Pa.) with data analysis using MetaMorph software (Universal Imaging Corp., Downingtown, Pa.).

To quantitate the average immunofluorescence per cell, the cell-based assay may be designed to account for changes in total cell number per well by quantitating cell number using the nuclear-stain DAPI, thus improving the accuracy of analysis. Variability in total cell number per well is a common occurrence in cellular screening due to the nonspecific activities of compounds being tested, i.e., compound induced morphology changes and compound-induced adhesion changes. These nonspecific activities appear as positive inhibition in the assay, even though they do not inhibit the autophosphorylation event.

For the assay, an image of the cells is acquired at both the DAPI wavelength (365 nm excitation, 405 nm emission) and the Alexafluor-488 wavelength (485 nm excitation, 535 mm emission). The DAPI image is thresholded to separate the fluorescence signal from the background signal and the total area for the fluorescence signal is measured. In the present context, “thresholded” means the process of defining a specific intensity level for determining which of two values will be assigned to each pixel in binary processing. If the pixel's brightness is above the threshold level, it will appear white in the image itself, or in the electronic image map; if below the threshold level, it will be designated as, or appear, black. The total DAPI fluorescence area is then divided by the average area per single cell to calculate the total cell number per well (TCPW). The Alexafluor image is subsequently thresholded to separate the fluorescence signal from the background signal and the total intensity for the fluorescence signal per well is measured (TFPW). By dividing the total fluorescence per well (TFPW) by the total cell number per well (TCPW), an average fluorescence per well is calculated which is independent of total cell number per well.

For high-throughput applications, candidate inhibitors may be screened using a conventional plate reader, with appropriate controls allowing for subtraction of background signal intensity. The high-throughput plate reader screen can be combined with the image-based assay approach to rapidly and accurately identify inhibitors.

To determine the effects of known kinase inhibitors on activated KIT, HEK293 cells stably expressing KITD816V, or KITY823F, as well as HEK cells containing the pcDNA3.1 vector, were seeded in 96-well plates and cultured with varying concentrations of staurosporin or DMSO as described above. Consistent with conventional practice in the field, controls for variation in cell number and fluorescent background signal arising from non-specific binding of labeled secondary antibody were typically included in the assay. Total signal intensity readings of pY823/anti-rabbit IgG-stained cells were obtained as described above, using cellular imaging system and ANALYST™ with close IC₅₀ and Z′ factor values to measure assay quality. For high-throughput screening assays, the lower the IC₅₀ value, the more sensitive the assay. The Z′ factor is a statistical value based on the signal-to-noise ratio and the difference between minimum and maximum luminescence readings. A Z′ factor of 1.0 indicates a perfect cell-based assay, e.g., one with essentially no variability in the control wells and background wells. A Z′ factor value greater than 0.5 indicates excellent assay quality.

As expected, no receptor phosphorylation was detected in KITY823F or pcDNA3.1-containing cells. HEK cells expressing KITD816V stained brightly for KIT phosphorylation, which was blocked by incubation with staurosporin. These results indicate that the cell-based assay provides a sensitive assay for detecting activated KIT receptors.

The number of cells at seeding determines the degree of cell confluency at the time of inhibitor addition and staining, which affects the reproducibility of the assay result. To determine the optimal cell-seeding quantity, KITD816V expressing cells were seeded in duplicate in 384-well plates at 2×10⁵, 2.5×10⁵, 3×10⁵, or 3.5×10⁵ cells/well and grown at 37° C. IC₅₀ and Z′ factors were measured at each concentration. IC₅₀ values were similar at 2.5×10⁵, 3×10⁵ and 3.5×10⁵ cells/well; 3.5×10⁵ cells/well, which demonstrated the optimal Z′ factor (Z′=0.74), was chosen as the cell-seeding quantity for subsequent experiments.

### EXAMPLE 5

### High-Throughput Assays Detect Inhibition of Constitutively Active KIT Mutant in Response to KIT Inhibitors

To determine the effects of a clinically useful KIT modulator, i.e., a KIT inhibitor, on the blockade of constitutively active KIT phosphorylation, HEK cells expressing mutant KIT receptors were prepared for analysis in the cell-based, high-throughput assay described above and cultured in the presence of an inhibitor (SU6577) of KIT activity. SU6577 is an indolinone compound previously characterized as an inhibitor of KIT related receptors, PDGF-R and VEGF-R. Intracellular levels of receptor activity were measured as a direct response to inhibitor.

KITΔK550-558-expressing HEK293 cells were seeded in duplicate onto 96-well, black, clear-bottom plates as described above. Cells were cultured 30 minutes with varying concentrations of AS701932/1 (SU6577) or DMSO prediluted in growth medium. Total signal intensity was obtained using a cellular imaging system and Analyst™, as described above. Cells expressing KITΔK550-558 demonstrated staining in the presence of the pY823 antibody, which was abolished by treatment with SU6577. Cells exhibited an IC₅₀ of 0.3 μM and Z′ factors of 0.8 were achieved.

These results demonstrate that the cell-based assay disclosed herein is effective at measuring KIT receptor tyrosine phosphorylation in multiple types of constitutively active KIT mutants and provides a sensitive method for high-throughput screening for inhibitors of constitutively activated KIT mutants.

### EXAMPLE 6

### Characterization of KIT Gain-of-Function Mutants

To further confirm that KIT gain-of-function mutations typically are constitutively active, HEK293 cells were transfected as in Example 1 with known gain-of-function mutants, such as ΔK550-558, ΔV559-560 and V559D derived from GISTs, KITD816V mutant commonly found in mastocytosis and AML patients, and the KITD816H mutant commonly found in germ cell tumors.

The transfected 293 cells were stimulated with KIT ligand, lysed and KIT receptor immunoprecipitates were analyzed using phosphotyrosine antibodies to determine the activation state of the receptor. SDS-PAGE and immunoblot revealed that all gain-of-function mutant receptors tested demonstrate high levels of tyrosine phosphorylation independent of Stem Cell Factor induction.

To determine the effects of the D816V activation loop gain-of-function mutation on KIT cell-surface expression, fluorescence microscopy was performed as described in Example 2. Analysis of the KITD816V intracellular localization revealed that the mutant receptor protein is retained within the cell and is not expressed on the cell-surface, unlike wild-type KIT receptor. Intracellular localization of KITD816V implied that the protein may be retained in the endoplasmic reticulum (ER).

Newly synthesized proteins are folded in the ER with the help of chaperone proteins, which assist in correct protein folding and prevention of protein aggregation. One chaperone pathway, the calnexin/calreticulin pathway, also functions as a component of the ER quality control system, identifying misfolded proteins and retaining these proteins in the ER. Calnexin and calreticulin are well characterized immunofluorescent markers of the ER compartment.

To determine if the KITD816V mutant was retained in the ER, immunofluorescence was performed. Transfected 293 cells were stained for KIT receptor and ER markers calnexin and calreticulin [Stressgen Biotechnologies Corp., Victoria, BC Canada (catalog #SPA-600 and cat# SPA-850)]. Assessment of the intracellular location of the D816V mutation showed that the mutant KITD816V co-localizes with calnexin and calreticulin in the ER.

These results indicate that KITD816V activation loop gain-of-function mutant is retained in the ER and not expressed on the cell surface.

### EXAMPLE 7

### Identification of Additional KIT Inhibitors Using the Cell-Based Screening Assay

Tatton et al. (J. Biol. Chem. 278:4847-53, 2003) disclosed that Src tyrosine kinase inhibitors, i.e., the pyrazolo-pyrimidine compounds PP1 and PP2, also act as inhibitors of activated KIT receptors, including KITD814V and KITD814Y expressed in transfected cells. To determine if the PP1 and PP2 compounds effectively inhibit active KIT receptors, HEK293 cells stably transfected with D816V mutant KIT receptor were cultured with PP1 and PP2 over a range of concentrations, as described above. After incubation with the inhibitor, the cells were immunostained with pY823 antibodies and analyzed as described in Example 4.

Data analysis showed that PP1 and PP2 block constitutive activation of the KITD816V mutant, with each compound exhibiting an IC₅₀ of approximately 1 μM. These results confirm that PP1 and PP2 are effective inhibitors of constitutively active KITD816V receptor and validate the immunofluorescence method as being a viable tool for identifying KitD816V inhibitors.

To identify and determine the ability of potential KIT inhibitors to prevent KIT activation in cells that naturally express mutant KIT receptors, the cell-based cell-viability assay was performed using human-mast cell line HMC1.1 (naturally expressing KITV560G), HMC1.2 (naturally expressing KITV560G and KITD816V mutations), and a P815 murine mastocytoma cell line (naturally expressing KITD814V, a murine analog of KITD816V). The cell-viability assay was performed using an ATPlite™ assay (PerkinElmer Life Sciences, Boston, Mass.), according to the manufacturer's protocol. Briefly, cells were plated 2×10³ cells/well in 96 well plates in RPMI 1640 medium supplemented with 10% fetal calf serum. Cells were incubated for 72 hours in the presence or absence of KIT inhibitors and cell viability measured

HMC1.1 and HMC1.2 cells incubated with Staurosporine (IC₅₀=20 nM) and Gleevec (IC₅₀=60 nM) demonstrated a concentration-dependent inhibition of proliferation. For HMC1.1 cells, Staurosprine showed 20% inhibition at 10⁻² μM and 100% inhibition at 10⁻¹ μM, while Gleevec showed approximately 80% inhibition at 10⁻¹ μM. For HMC1.2 cells, Staurosporine (IC₅₀=6 nM) demonstrated 70% inhibition at 10⁻² μM and approximately 97% inhibition at 10⁻¹ μM, while Gleevec did not inhibit proliferation of HMC1.2 cells at any concentration. These results indicate that occurrence of D816V mutation in the KIT receptor confers resistance to Gleevec inhibition of KIT-induced proliferation in mast cell leukemia HMC1.2 cells. HMC1.1 and 1.2 sublines provide a useful system to assess the efficiency of KIT inhibitors, and identify KIT juxtamembrane mutant inhibitors vs. KITD816V activation loop inhibitors.

P815 cells expressing D814V were cultured with KIT inhibitor AS396773 (SU396773) (U.S. Pat. No. 5,792,783), a substituted indolinone, and assayed for inhibition of KIT-induced proliferation. AS396773 (IC₅₀ 500 nM) demonstrated approximately 92% inhibition of P815 cell proliferation at a concentration of approximately 2 μM. AS396773 was also shown by immunofluorescence microscopy to inhibit KIT receptor phosphorylation in cells expressing D816V and ΔK550-558.

These results validate the P815 proliferation assay in combination with the immunofluorescence method as viable tools for identifying KitD816V inhibitors. These results also indicate that, in addition to detecting KIT inhibition in transfected non-mastoid cells, the cell-based assay is effective at identifying KIT inhibitors in mast cells endogenously expressing a mutant KIT receptor.

### EXAMPLE 8

### KIT D816V Induces Tyrosine Phosphorylation and Nuclear Translocation of STAT5

KIT receptor is known to induce downstream phosphorylation in cells after binding its ligand, SCF. One downstream factor induced by activated KIT is Stat5 (Signal Transducers and Activators of Transcription 5), which is activated by tyrosine phosphorylation. STATs comprise a family of cytoplasmic transcription factors that transmit signals to the nucleus where STATs bind to specific DNA promoter sequences regulating gene expression (Darnell et al., Science 264:1415-1421, 1994). STAT signaling is critical for many normal cellular processes, and studies have shown that aberrant STAT signaling by constitutively active Stat proteins, in particular Stat3 and Stat5, participate in the development and progression of human cancers by either preventing apoptosis, inducing cell proliferation, or both (Bowman et al., Oncogene, 19:2474-88, 2000). Stat5 is activated by the synergistic effects of erythropoietin (EPO) and SCF binding to cell-surface receptors, resulting in the induction of Stat5 translocation to the nucleus. Recent studies indicated that SCF alone cannot induce Stat5 translocation (Boer et al., Exp. Hematol. 31:512-20, 2003).

To determine if constitutively active KIT receptor can induce activation of Stat5, intracellular localization of endogenous Stat5 was assayed in HEK293 cells transiently transfected with constitutively active KITD816V. Cells expressing low levels of KITD816V induced translocation of Stat5 into the nucleus

Immunoprecipitation with STAT5 antibody from HEK293 cells transiently transfected with KITD816V and Western blot analysis using antibody to pY694 Stat5 showed that that KITD816V overexpression causes phosphorylation of endogenous Stat5 on Y694.

Taken together these results demonstrate that constitutively activated KIT can induce Stat5 activation in an SCF-independent manner, suggesting another readout for inhibitors of constitutively activated KIT receptor mutants.

To determine if KIT inhibitors work by preventing translocation of Stat5 to the nucleus, HEK293 cells transfected with KITD816V were incubated with staurosporine (1 μM and AS396773 (10 μM) and assessed for Stat5 translocation by immunoprecipitation and immunoblot. Western blot showed that both Staurosporine and AS396773 inhibited Stat5 translocation by constitutively active KIT receptor.

These results demonstrate that in addition to the cell-based assay to identify KIT inhibitors, Stat5 is a useful marker to indicate that a candidate inhibitor prevents phosphorylation of KIT and acts as a KIT inhibitor.

### EXAMPLE 9

### Solution-Based Measurement of KIT Phosphorylation in Response to Inhibitor

The cell-based assay described above, in which KIT mutant-expressing cells are bound on a solid support, can also be carried out using a solution-based assay. The ability to carry out this assay in solution allows for measurement of KIT receptor activation in a broader range of cell types, e.g., not necessarily adherent cells. The solution-based assay also facilitates the measurement of cells isolated from patients expressing a mutant KIT receptor, independent of the cell type containing the mutation.

Solution-based measurement of KIT tyrosine phosphorylation is assessed by intracellular flow cytometry (Jung et al., J. Immunol. Methods 173:219-228, 1993). HEK293 cells or cells of any other cell line expressing a mutant KIT are placed in a 96-well, round-bottom plate at a concentration appropriate for flow cytometric analysis, e.g. at least 1×10⁶ or 2×10⁶ cell/well. This cell concentration is optimized for the antibody and the cell type using routine experimentation. Mutant KIT-expressing cells are either stimulated with SCF or cultured with a candidate KIT modulator, such as an inhibitor, as described above, to modulate intracellular KIT tyrosine phosphorylation.

After culture in the presence of SCF or inhibitor for a period of time, KIT-expressing cells are contacted with a phospho-KIT specific antibody. Cells are isolated by centrifugation to remove media and inhibitor, and resuspended and washed with staining buffer (PBS containing 2% goat serum, 0.5% BSA, 2 mM EDTA, optional Azide). Cells are then resuspended in 100 μL 4% paraformaldehyde and fixed 20 minutes at 4° C. in the dark. Cells are washed by centrifugation and resuspension 2 times in staining buffer and resuspended in 100 μL of PBS containing 1% saponin to permeabilize the cells. Cells are allowed to permeabilize for 15 minutes at 4° C. Cells are then washed 2× in staining buffer and resuspended in 100 μL staining buffer. Cells are then stained, as described above, with KIT antibodies and/or pY823 to detect activated KIT receptor. Cells are washed again and resuspended in an appropriate volume staining buffer, from 200 μL to 0.5 ml depending on the cell number to be detected, for flow cytometric analysis. Cell fixation and permeabilization may also be carried out with commercially available reagents according to the manufacturer's protocol (Cytofix/Cytoperm™ reagents, Pharmingen, Inc., San Diego, Calif.). Staining of surface antigens, i.e., cell-specific markers or morphological markers, is carried out before the fixation step and will be stained with antibodies exciting in a different channel than the KIT-specific or phosphotyrosine antibodies. Multicolor flow cytometric analysis is carried out on a flow cytometer such as a FACScan® or a FACScalibur® (Becton Dickinson), according to the manufacturer's protocol.

In one embodiment, HEK cells stably transfected with vectors expressing KITWT, KITD816V, KITΔK550-558 or any other clinically relevant KIT mutant are cultured with a candidate modulator (e.g., inhibitor) and harvested for flow cytometry as described herein. A decrease in signal derived from pY823 antibody in D816V-expressing cells or KITΔK550-558-expressing cells indicates that a candidate modulator that inhibits auto-phosphorylation effectively blocks a constitutively active KIT receptor, and is a candidate for a useful therapeutic in the treatment of mastocytosis and other mast cell diseases.

In other embodiments, cells are isolated from a patient exhibiting a mast cell disorder, leukemia originating from a mast cell disease, or other related tumor and prepared for KIT receptor analysis by flow cytometry. The benefit of the flow cytometric method is that numerous cell types, both adherent and non-adherent cell types, are adaptable to staining by intracellular flow cytometry. This allows for detection of constitutively active KIT receptor mutants directly from patients demonstrating a mast cell disorder, and analysis of the receptor's susceptibility to inhibition by a candidate inhibitor.

A decrease in tyrosine phosphorylation as a result of exposure of cells known or suspected to be expressing an activated KIT receptor, including ligand-independent activated KIT, to a candidate inhibitor indicates that a particular inhibitor is useful for treating that specific patient. These cell-based assays provide a versatile therapeutic approach that can be personally tailored to the treatment of patients with particular mast cell disorders, by identifying a KIT modulator (e.g., inhibitor) that is specifically effective in preventing an individual patient's disease, or ameliorating at least one symptom associated therewith.

### EXAMPLE 10

### Treatment of Mast Cell Disorders Using Inhibitors Identified in a Cell-Based, High-Throughput Screen

Treatments for mast cell disorders are typically based on non-specific kinase inhibitors or other treatments designed to treat cancer-related diseases. For example, mastocytosis is often treated with Gastrocrom (Aventis, Inc.), but this reagent is not effective against advanced forms of the disease. Leukemias related to mast cell disorders are treated with non-specific treatments, e.g., AML is often treated with a DNA synthesis inhibitor, such as cytarabine or anthrocycline. Germ cell tumors, which exhibit the KITD816H mutation, are often treated with therapeutics lacking specific targeting such as bleomycin or cis-platin, possibly causing lung toxicity or kidney damage.

Inhibitors identified as effective against a mutant KIT receptor in the high-throughput screen may be developed into a pharmaceutical composition for administration to a subject in need thereof. The KIT inhibitors may be administered by any route appropriate for administration, depending on the type of mast cell disorder being treated. An inhibitor identified by the present screening method may be administered in conjunction with other therapies for treatment of mast cell disorders or cancers. Mast cell disorders or cancers characterized by aberrant mast cell proliferation that are amenable to treatment with KIT inhibitors identified by a screening method according to the invention include, but are not limited to, mastocytosis, mast cell leukemia, mast cell sarcoma, a germ cell tumor, a gastrointestinal stromal tumor, an acute myeloid leukemia (AML), a chronic myeloid leukemia (CML), a chronic myelomonocytic leukemia (CMML), a sinonasal lymphoma, an ovarian tumor, a breast tumor, a small lung cell carcinoma, a neuroblastoma, and a melanoma.

An inhibitor identified by a screening method may be administered to a patient in need, as described in Ryan et al. (Oncologist 7:531-38, 2002). The inhibitor is administered in doses appropriate for the patient's size, sex, and weight, e.g., at a target dose of 1.5 mg/m², 400 mg, 800 mg, or other appropriate dose, as would be known or readily determined in the art. Subsequent doses of the inhibitor may be increased or decreased to address the particular patient's response to therapy. Patients can receive escalating doses of KIT receptor inhibitor until the maximum tolerated dose (MTD) is determined. The MTD is defined as the dose preceding that at which an established fraction of recipients, experience dose-limiting toxicity, such as at least 2 of 3 or 2 of 6 patients.

The inhibitor may be administered continuously, e.g., through intravenous delivery or by slow release methods, for an extended period of time. The administration may last 4-24 hours, or longer and is amenable to optimization using routine experimentation. The inhibitor may also be given for a duration not requiring extended treatment. Additionally, the inhibitor may be administered daily, weekly, bi-weekly, or at other frequencies, as would be determinable by one of ordinary skill in the art.

In one approach, the effectiveness of treatment is determined by computer tomographic (CT) scans of the tumor area with the degree of tumor regression assessed by measuring the decrease in tumor size. Biopsies or blood samples are also used to assess the presence or absence of particular cell types in response to treatment with the KIT receptor inhibitor. These response assessments are made periodically during the course of treatment to monitor the response of a patient to a given therapy.

Gastrointestinal stromal tumors (GISTs) are associated with several constitutively activated KIT receptor mutants. Patients demonstrating GISTs are treated with KIT inhibitors identified by a screening method of the invention. An appropriate dose of KIT inhibitor as determined by the treating physician, is administered as described previously. An exemplary treatment dose may include a range of 250 mg up to 1000 mg KIT inhibitor daily. A range of 400-600 mg daily has been used in the administration of Gleevec. Patients may receive inhibitor twice daily, and treatment may continue for one week up to one month, or up to two months. The therapeutics may also be administered at weekly intervals or biweekly intervals. Therapeutics may be re-administered as necessary.

Efficacy of KIT inhibitor therapy is measured in patients exhibiting a GIST based on improvement in tumor grade toxicity or based on reduced rate of tumor progression, as assessed by measurement of tumor grade toxicity, tumor size and mitotic cell count (Strickland et al., Cancer Control, 8: 252-261, 2001; Fletcher et al., Human Pathology 33:459-465, 2002). GISTs are scored on a scale of Grade 1-4, with 4 being the most severe tumor type. The grades are based on morphological indications, tumor size, and cell counts. Patients are assessed for a decrease in tumor score as well as a decrease in mitotic cell numbers, which are indicative of a decrease in dividing, tumorigenic cells.

An improvement in tumor score and patient prognosis after treatment with a KIT inhibitor identified by a method of the invention indicates that the screening method identifies compounds capable of effectively treating patients having a GIST, such as by decreasing the severity of a symptom associated with the disease. KIT inhibitors identified by the methods disclosed herein are expected to be therapeutically useful in the treatment of other cancers characterized by aberrant KIT tyrosine kinase receptor expression.

It is contemplated that the KIT receptor inhibitor will be administered alone or in conjunction with other chemotherapeutics, as well as with treatments designed to decrease any side effect of a particular treatment regimen.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

We claim:

1. A method of screening for an inhibitor of an active KIT tyrosine kinase receptor in a cell comprising:
(a) contacting a cell comprising an active KIT tyrosine kinase receptor with a candidate inhibitor; and
(b) detecting KIT activity by using a phosphotyrosine-specific antibody to determine the amount of KIT tyrosine phosphorylation in the presence and in the absence of said inhibitor,
wherein a decrease in KIT tyrosine phosphorylation in the presence of said candidate inhibitor in comparison to the KIT tyrosine phosphorylation in its absence identifies the candidate inhibitor as a KIT inhibitor.

2. The method according to claim 1 wherein said KIT tyrosine kinase receptor is constitutively active.

3. The method according to claim 2 wherein the constitutively active KIT tyrosine kinase receptor has a mutation in the phosphotransferase tyrosine kinase domain.

4. The method according to claim 3 wherein the mutation is in the activation loop of the KIT tyrosine kinase domain.

5. The method according to claim 2 wherein the constitutively active KIT tyrosine kinase receptor has a mutation in the juxtamembrane domain.

6. The method according to claim 5 wherein the mutation is a deletion of amino acids 550-558 of SEQ ID NO:2.

7. The method according to claim 2 wherein the constitutively active KIT tyrosine kinase receptor has a mutation in the extracellular domain.

8. The method according to claim 7 wherein the mutation is a substitution mutation of AY502-503 in SEQ ID NO: 2.

9. The method according to claim 1 wherein the KIT tyrosine kinase receptor comprises an amino acid sequence selected from the group consisting of SEQ ID NOS:2, 4, and 6

10. The method according to claim 9 wherein the KIT tyrosine kinase receptor comprises the amino acid sequence set forth in SEQ ID NO:2.

11. The method according to claim 4 wherein the KIT tyrosine kinase receptor comprises a substituted amino acid at position 816 of SEQ ID NO:2.

12. The method according to claim 11 wherein the substituted amino acid is selected from the group consisting of Valine, Histidine, Phenylalanine, Tyrosine, or Glycine.

13. The method according to claim 12 wherein the substituted amino acid is Valine.

14. The method according to claim 1 wherein the cell comprising the active KIT tyrosine kinase receptor is bound to a solid support.

15. The method according to claim 14 further comprising detecting cellular morphology, cytoskeletal rearrangement, or nuclear staining of said cell in the presence and in the absence of the candidate inhibitor.

16. The method according to claim 1 wherein the phosphotyrosine-specific antibody is selected from the group consisting of a monoclonal antibody, a polyclonal antibody, a chimeric antibody, a humanized antibody, a single-chain antibody, and an antibody fragment.

17. The method according to claim 16 wherein the phosphotyrosine-specific antibody is pY823.

18. The method according to claim 16 wherein the phosphotyrosine-specific antibody binds to an auto-phosphorylation site of said KIT tyrosine kinase receptor.

19. The method according to claim 16 wherein the phosphotyrosine-specific antibody is detectably labeled.

20. The method according to claim 19 wherein the detectable label is a fluorophore or a radiolabel.

21. The method according to claim 1 wherein the detecting step comprises flow cytometry.

22. The method according to claim 1 wherein the active KIT tyrosine kinase receptor is expressed from a heterologous vector.

23. The method according to claim 1 wherein the active KIT tyrosine kinase receptor is endogenous to the cell.

24. The method according to claim 23 wherein the cell is isolated from a tumor.

25. The method according to claim 24 wherein the tumor is selected from the group consisting of a mast cell leukemia, mast cell sarcoma, a germ cell tumor, a gastrointestinal stromal tumor, an acute myeloid leukemia (AML), a chronic myeloid leukemia (CML), a chronic myelomonocytic leukemia (CMML), a sinonasal lymphoma, an ovarian tumor, a breast tumor, a small lung cell carcinoma, a neuroblastoma, and a melanoma.

26. A kit for screening for an inhibitor of active KIT tyrosine kinase receptor comprising a phosphotyrosine antibody and instruction for performing a screen according to claim 1 for said inhibitor.

27. A method of treating a condition selected from the group consisting of mastocytosis, mast cell leukemia, mast cell sarcoma, a germ cell tumor, a gastrointestinal stromal tumor, an acute myeloid leukemia (AML), a chronic myeloid leukemia (CML), a chronic myelomonocytic leukemia (CMML), a sinonasal lymphoma, an ovarian tumor, a breast tumor, a small lung cell carcinoma, a neuroblastoma, and a melanoma, comprising administering an inhibitor identified according to the method of claim 1.

28. A method for designing a treatment regimen for a patient with a mast cell disorder comprising:
(a) isolating a cell from said patient, wherein said cell comprises an active KIT tyrosine kinase receptor;
(b) contacting said cell with a KIT inhibitor identified by the method of claim 1;
(c) detecting KIT activity in said cell using a phosphotyrosine-specific antibody to determine the amount of KIT tyrosine phosphorylation in the presence and in the absence of said inhibitor; and
(d) designing a treatment regimen for said patient which includes administration of the KIT inhibitor that specifically inhibits KIT activity in said patient.


SEQUENCE LISTING (6 sequences)

SEQ ID NO: 1 - DNA, 5084 bp, Homo sapiens
  CDS (22)..(2952)
gatcccatcgcagctaccgcgatgagaggcgctcgcggcgcctgggattttctctgcgttctgctcctactgcttcgcgtccagacaggctcttctcaaccatctgtgagtccaggggaaccgtctccaccatccatccatccaggaaaatcagacttaatagtccgcgtgggcgacgagattaggctgttatgcactgatccgggctttgtcaaatggacttttgagatcctggatgaaacgaatgagaataagcagaatgaatggatcacggaaaaggcagaagccaccaacaccggcaaatacacgtgcaccaacaaacacggcttaagcaattccatttatgtgtttgttagagatcctgccaagcttttccttgttgaccgctccttgtatgggaaagaagacaacgacacgctggtccgctgtcctctcacagacccagaagtgaccaattattccctcaaggggtgccaggggaagcctcttcccaaggacttgaggtttattcctgaccccaaggcgggcatcatgatcaaaagtgtgaaacgcgcctaccatcggctctgtctgcattgttctgtggaccaggagggcaagtcagtgctgtcggaaaaattcatcctgaaagtgaggccagccttcaaagctgtgcctgttgtgtctgtgtccaaagcaagctatcttcttagggaaggggaagaattcacagtgacgtgcacaataaaagatgtgtctagttctgtgtactcaacgtggaaaagagaaaacagtcagactaaactacaggagaaatataatagctggcatcacggtgacttcaattatgaacgtcaggcaacgttgactatcagttcagcgagagttaatgattctggagtgttcatgtgttatgccaataatacttttggatcagcaaatgtcacaacaaccttggaagtagtagataaaggattcattaatatcttccccatgataaacactacagtatttgtaaacgatggagaaaatgtagatttgattgttgaatatgaagcattccccaaacctgaacaccagcagtggatctatatgaacagaaccttcactgataaatgggaagattatcccaagtctgagaatgaaagtaatatcagatacgtaagtgaacttcatctaacgagattaaaaggcaccgaaggaggcacttacacattcctagtgtccaattctgacgtcaatgctgccatagcatttaatgtttatgtgaatacaaaaccagaaatcctgacttacgacaggctcgtgaatggcatgctccaatgtgtggcagcaggattcccagagcccacaatagattggtatttttgtccaggaactgagcagagatgctctgcttctgtactgccagtggatgtgcagacactaaactcatctgggccaccgtttggaaagctagtggttcagagttctatagattctagtgcattcaagcacaatggcacggttgaatgtaaggcttacaacgatgtgggcaagacttctgcctattttaactttgcatttaaaggtaacaacaaagagcaaatccatccccacaccctgttcactcctttgctgattggtttcgtaatcgtagctggcatgatgtgcattattgtgatgattctgacctacaaatatttacagaaacccatgtatgaagtacagtggaaggttgttgaggagataaatggaaacaattatgtttacatagacccaacacaacttccttatgatcacaaatgggagtttcccagaaacaggctgagttttgggaaaaccctgggtgctggagctttcgggaaggttgttgaggcaactgcttatggcttaattaagtcagatgcggccatgactgtcgctgtaaagatgctcaagccgagtgcccatttgacagaacgggaagccctcatgtctgaactcaaagtcctgagttaccttggtaatcacatgaatattgtgaatctacttggagcctgcaccattggagggcccaccctggtcattacagaatattgttgctatggtgatcttttgaattttttgagaagaaaacgtgattcatttatttgttcaaagcaggaagatcatgcagaagctgcactttataagaatcttctgcattcaaaggagtcttcctgcagcgatagtactaatgagtacatggacatgaaacctggagtttcttatgttgtcccaaccaaggccgacaaaaggagatctgtgagaataggctcatacatagaaagagatgtgactcccgccatcatggaggatgacgagttggccctagacttagaagacttgctgagcttttcttaccaggtggcaaagggcatggctttcctcgcctccaagaattgtattcacagagacttggcagccagaaatatcctccttactcatggtcggatcacaaagatttgtgattttggtctagccagagacatcaagaatgattctaattatgtggttaaaggaaacgctcgactacctgtgaagtggatggcacctgaaagcattttcaactgtgtatacacgtttgaaagtgacgtctggtcctatgggatttttctttgggagctgttctctttaggaagcagcccctatcctggaatgccggtcgattctaagttctacaagatgatcaaggaaggcttccggatgctcagccctgaacacgcacctgctgaaatgtatgacataatgaagacttgctgggatgcagatcccctaaaaagaccaacattcaagcaaattgttcagctaattgagaagcagatttcagagagcaccaatcatatttactccaacttagcaaactgcagccccaaccgacagaagcccgtggtagaccattctgtgcggatcaattctgtcggcagcaccgcttcctcctcccagcctctgcttgtgcacgacgatgtctgagcagaatcagtgtttgggtcacccctccaggaatgatctcttcttttggcttccatgatggttattttcttttctttcaacttgcatccaactccaggatagtgggcaccccactgcaatcctgtctttctgagcacactttagtggccgatgatttttgtcatcagccaccatcctattgcaaaggttccaactgtatatattcccaatagcaacgtagcttctaccatgaacagaaaacattctgatttggaaaaagagagggaggtatggactgggggccagagtcctttccaaggcttctccaattctgcccaaaaatatggttgatagtttacctgaataaatggtagtaatcacagttggccttcagaaccatccatagtagtatgatgatacaagattagaagctgaaaacctaagtcctttatgtggaaaacagaacatcattagaacaaaggacagagtatgaacacctgggcttaagaaatctagtatttcatgctgggaatgagacataggccatgaaaaaaatgatccccaagtgtgaacaaaagatgctcttctgtggaccactgcatgagcttttatactaccgacctggtttttaaatagagtttgctattagagcattgaattggagagaaggcctccctagccagcacttgtatatacgcatctataaattgtccgtgttcatacatttgaggggaaaacaccataaggtttcgtttctgtatacaaccctggcattatgtccactgtgtatagaagtagattaagagccatataagtttgaaggaaacagttaataccattttttaaggaaacaatataaccacaaagcacagtttgaacaaaatctcctcttttagctgatgaacttattctgtagattctgtggaacaagcctatcagcttcagaatggcattgtactcaatggatttgatgctgtttgacaaagttactgattcactgcatggctcccacaggagtgggaaaacactgccatcttagtttggattcttatgtagcaggaaataaagtataggtttagcctccttcgcaggcatgtcctggacaccgggccagtatctatatatgtgtatgtacgtttgtatgtgtgtagacaaatatttggaggggtatttttgccctgagtccaagagggtcctttagtacctgaaaagtaacttggctttcattattagtactgctcttgtttcttttcacatagctgtctagagtagcttaccagaagcttccatagtggtgcagaggaagtggaaggcatcagtccctatgtatttgcagttcacctgcacttaaggcactctgttatttagactcatcttactgtacctgttccttagaccttccataatgctactgtctcactgaaacatttaaattttaccctttagactgtagcctggatattattcttgtagtttacctctttaaaaacaaaacaaaacaaaacaaaaaactccccttcctcactgcccaatataaaaggcaaatgtgtacatggcagagtttgtgtgttgtcttgaaagattcaggtatgttgcctttatggtttcccccttctacatttcttagactacatttagagaactgtggccgttatctggaagtaaccatttgcactggagttctatgctctcgcacctttccaaagttaacagattttggggttgtgttgtcacccaagagattgttgtttgccatactttgtctgaaaaattcctttgtgtttctattgacttcaatgatagtaagaaaagtggttgttagttatagatgtctaggtacttcaggggcacttcattgagagttttgtcttgccatactttgtctgaaaaattcctttgtgtttctattgacttcaatgatagtaagaaaagtggttgttagttatagatgtctaggtacttcaggggcacttcattgagagttttgtcaatgtcttttgaatattcccaagcccatgagtccttgaaaatattttttatatatacagtaactttatgtgtaaatacataagcggcgtaagtttaaaggatgttggtgttccacgtgttttattcctgtatgttgtccaattgttgacagttctgaagaattc

SEQ ID NO: 2 - protein, 976 aa, Homo sapiens [sequence omitted]

SEQ ID NO: 3 - DNA, 5132 bp, Mus musculus
  CDS (29)..(2956)
gagctcagagtctagcgcagccaccgcgatgagaggcgctcgcggcgcctgggatctgctctgcgtcctgttggtcctgctccgtggccagacagccacgtctcagccatctgcaagtccaggggagccgtctccgccatccatccatccagcacaatcagagttaatagttgaagctggcgacaccctcagcctgacgtgcattgatcccgactttgtcagatggactttcaagacctatttcaatgaaatggttgagaataaaaaaaatgaatggatccaggaaaaagccgaggccactcgcacgggcacatacacgtgcagcaacagcaatggcctcacgagttctatttacgtgtttgttagagatcctgccaaacttttcctggttggccttcccttgtttggcaaagaagacagcgacgcgctggtccgctgccctctgacagacccacaggtgtccaattattccctcatcgagtgtgatgggaaatctctccccacggacctgacgtttgtcccaaaccccaaggctggcatcaccatcaaaaacgtgaagcgcgcctaccaccggctctgtgtccgctgtgctgctcagcgtgacggtacatggctgcattctgacaaattcaccctcaaagtgcgggaagccatcaaggctatccctgttgtgtctgtgcctgaaacaagtcacctccttaagaaaggggacacatttacggtggtgtgcaccataaaagatgtgtctacatccgtgaactccatgtggctaaagatgaaccctcagcctcagcacatagcccaggtaaagcacaatagctggcaccggggtgacttcaattatgaacgccaggagacgctgactatcagctcggcaagagttgacgattctggagtgttcatgtgttatgccaataatacttttggatcagcaaatgtcacaacaaccttgaaagtagtagaaaaaggattcatcaacatctcccctgtgaagaacactacagtatttgtaaccgatggagaaaacgtagatttggttgttgaatacgaggcctaccccaaacccgagcaccagcagtggatatatatgaacaggacctcggctaacaaagggaaggattatgtcaaatctgataacaaaagcaacatcagatatgtgaaccaacttcgcctgaccagattaaaaggcacagaaggaggcacttatacctttctggtgtccaactctgatgccagtgcttccgtgacattcaacgtttacgtgaacacaaaaccagaaatcctgacgtacgacaggctcataaatggcatgctccagtgtgtggcagagggattcccggagcccacaatagattggtatttttgtacaggagcagagcaaaggtgtaccactcctgtctcaccagtggacgtacaggtccagaatgtatctgtgtcaccatttggaaaactggtggttcagagttccatagactccagcgtcttccggcacaacggcacggtggagtgtaaggcctccaacgatgtgggcaagagttccgccttctttaactttgcatttaaagagcaaatccaggcccacactctgttcacgccgctgctcattggctttgtggtcgcagctggcgcgatggggatcattgtgatggtgctcacctacaaatatttgcagaaacccatgtatgaagtacaatggaaggttgtcgaggagataaatggaaacaattatgtttacatagacccgacgcaacttccttatgatcacaaatgggagtttcccagaaacaggctgagttttggaaagacattgggagctggtgccttcgggaaggtcgttgaggccactgcatatggcttgattaagtcggatgctgccatgacagttgccgtgaagatgctcaaaccaagtgcccatttaacagaaagagaggccctaatgtcggaactgaaggtcctgagctacctgggcaatcacatgaatattgtgaacctgcttggcgcatgcacggtgggagggcccaccctggtcattacagaatattgttgctatggtgatcttttgaattttttgagaaggaagcgtgactcgtttattttctcaaagcaagaagagcaggcagaagcggcactttataagaaccttctgcactcaacggagccttcctgtgacagttcaaatgaatatatggacatgaagcctggcgtttcctacgtggtgccaaccaagacagacaagaggagatccgcaagaatagactcgtacatagaaagagacgtgactcctgccatcatggaagatgacgagctggctctggacctggatgatttgctgagcttctcctaccaggtggccaaggcgatggcgttcctcgcctccaagaattgtattcacagagatttggcagccaggaatatcctcctcactcacgggcggatcacaaagatttgcgatttcgggctagccagagacatcaggaatgattcgaattacgtggtcaaaggaaatgcacgactgcccgtgaagtggatggcaccagagagcattttcagctgcgtgtacacatttgaaagtgatgtctggtcctatgggattttcctctgggagctcttctccttaggaagcagcccctacccagggatgccggtcgactccaagttctacaagatgatcaaggaaggcttccggatggtcagcccggagcacgcgcctgccgaaatgtatgacgtcatgaagacttgctgggacgctgaccccttgaaaaggccaacattcaagcaggttgtccaacttattgagaagcagatctcggacagcaccaagcacatttactccaacttggcaaactgcaaccccaacccagagaaccccgtggtggtggaccattccgtgagggtcaactcggtgggcagcagcgcctcttctacgcagcccctgctcgtgcacgaagatgcctgagcagaaacccaagtccaacaggctttgctgctgtctccgaccccgtccttctggcttctgtgatggttacttggtttccctttgacttgcatcctattccagggtagcgagttccccaccccacctccaaccccactgtgattccgcctttacgagcacacactttagtggccgatggcttttcttttctgccatcagccaccgtcccgctgcgaaggtccgaactgtatgtatatattttcccaatagcaaagtagctcctactgtaaacagaaggactcctcctgctttagaggagaagggaagggcggggtgaaactggatgcccagagttcttcccccagtgctcccctgagtgtatttgaaaagtatggccagtagttcacttgaagaatagatgtagtcccatttggccctgagagccatccttaatgatgggagatatatgtagcaagactagaaagccaagccctttgtgtagaaagcagaccattcttagaacagagggcaacggggcatcggaagtctggtcacgctaagaagaccgaggctgagaaggaacaagccaggggaagcgtgaacaatgatgctctgctctgggctgccgctcgggcttctgtacaactgacctggtttctcagtactttgctgtctgggagtagcattggaatcaaggcctcctccctagtcagcctttgtatatactcatctatacgttgtatgcgttcatactttggaggagggatttcccacaagctttcgtttctgtgtacagccctggattagacctactgtgtgtaagaatagattaagagccatacatatttgaaggaaacagttaaatgttttttggttgtggttgttgttgttgttgttttaaagaaaaaaatgtatatgctaagcacaatctttataagacctcttagccaacatacttgctctgtctacacttcggaacaagccttccatgtcagagtggctttgcaggcaggagaactgaggctgtttgaaaaggttaccacaggatggagaaaacagtgcagtcctggtttggattctcacatagcagggagcacaagttaaactcgaccttttataggcacgtcccggacatcgggcctgtatctattcaagtgtgtatgtgtgtgcatgcgtgtgtctatgcgtgtgggtgagttgtgttgggaaacttgccctgcatccctgagggtcctccttcaggacccaagacgtaacagcttctgtcaccgctcctgtctctccagtttccctgcatgtcgctcactgtctagaatttactcaaagccgccacagaggcttagcggagtgaagtgccgaaggacctctttatttggagtcctcctgtatttaacaacactcttatcgtagacccattcattagaccttatgtaatgctgccaatccagggaaacagatttaaagtgtaccccgtagacagggcccagaggttcccttgtccttgccctcccccacaccacccatgatcactgtccaacataaagggttcagtgtgttacgtggtcatgtgttgtccttacaggattcaggtatgttgccttcacggttttccccaccccctcctgccctttatcctttaggccgtgtggccatgaacctggaagaagtgatcgtttcgacttgagtgctacactcttgcacctttccaaagtaagctggtttggaggtcctgtggtcatgtacgagactgtcaccagttaccgcgctctgtttgaaacatgtctttgtattcctaatgacttcagttagagtaaggagaatagctgttaatatggatgtcaggtacttaaggggccacaccattgagaattttgtcttggatattcttgaaagtttatatttttataattttttttacatcagatgtcagatgtttctttcagttgcttgatgtttggaattattatgtggctttttttgtaaatattgaaatgtagcaataatgtcttttgaatattcctgagcccatgagtccctgaaaatattttttatatatacagtaactttatgtgtaaataatacgctgtgcaagtttaaacatgtcacgttacatgtgggttttttctgatatgttgtccaactgttgacagttctgaagaattctaataaaaatgtaaatatataaatcaaaaaaaaa

SEQ ID NO: 4 - protein, 975 aa, Mus musculus
  sig_peptide (1)..(21)
MRGARGAWDLLCVLLVLLRGQTATSQPSASPGEPSPPSIHPAQSELIVEAGDTLSLTCIDPDFVRWTFKTYFNEMVENKKNEWIQEKAEATRTGTYTCSNSNGLTSSIYVFVRDPAKLFLVGLPLFGKEDSDALVRCPLTDPQVSNYSLIECDGKSLPTDLTFVPNPKAGITIKNVKRAYHRLCVRCAAQRDGTWLHSDKFTLKVREAIKAIPVVSVPETSHLLKKGDTFTVVCTIKDVSTSVNSMWLKMNPQPQHIAQVKHNSWHRGDFNYERQETLTISSARVDDSGVFMCYANNTFGSANVTTTLKVVEKGFINISPVKNTTVFVTDGENVDLVVEYEAYPKPEHQQWIYMNRTSANKGKDYVKSDNKSNIRYVNQLRLTRLKGTEGGTYTFLVSNSDASASVTFNVYVNTKPEILTYDRLINGMLQCVAEGFPEPTIDWYFCTGAEQRCTTPVSPVDVQVQNVSVSPFGKLVVQSSIDSSVFRHNGTVECKASNDVGKSSAFFNFAFKEQIQAHTLFTPLLIGFVVAAGAMGIIVMVLTYKYLQKPMYEVQWKVVEEINGNNYVYIDPTQLPYDHKWEFPRNRLSFGKTLGAGAFGKVVEATAYGLIKSDAAMTVAVKMLKPSAHLTEREALMSELKVLSYLGNHMNIVNLLGACTVGGPTLVITEYCCYGDLLNFLRRKRDSFIFSKQEEQAEAALYKNLLHSTEPSCDSSNEYMDMKPGVSYVVPTKTDKRRSARIDSYIERDVTPAIMEDDELALDLDDLLSFSYQVAKAMAFLASKNCIHRDLAARNILLTHGRITKICDFGLARDIRNDSNYVVKGNARLPVKWMAPESIFSCVYTFESDVWSYGIFLWELFSLGSSPYPGMPVDSKFYKMIKEGFRMVSPEHAPAEMYDVMKTCWDADPLKRPTFKQVVQLIEKQISDSTKHIYSNLANCNPNPENPVVVDHSVRVNSVGSSASSTQPLLVHEDA

SEQ ID NO: 5 - DNA, 3816 bp, Rattus norvegicus
  CDS (45)..(2981)
gctgtagcagagagaggagctcagagtctagcgcagccaccgcgatgagaggcgctcgcggcgcctgggatctgctctgcgtcctgttggtcctgctccgtggccagacagggacttctcagccatctgcgagtccaggggagccgtctccaccatccatccagccggcccagtcagagttaatagttgaagccggggacaccatcaggctgacgtgcactgaccccgcctttgtcaaatggactttcgagatcctcgatgtaaggattgagaataagcagagcgaatggattcgagaaaaagccgaggccactcacacgggcaaatacacgtgcgtcagcggcagcggcctcaggagctctatttacgtgttcgttagagatcctgccgtacttttcctggttggccttcccttgtttggcaaagaagacaacgacgcactggtccgctgccccctgacagacccacaggtgtccaattactccctcattgagtgtgatgggaaatctctccccacggacctgaagttcgtccccaaccccaaggctggcatcaccatcaaaaacgtgaagcgcgcctaccaccggctgtgcatccggtgtgctgcccagcgtgagggcaaatggatgcggtctgacaaattcaccctcaaagtgagagcagccatcaaagctatcccagtggtgtctgtgcccgaaacaagtcatctccttaaggaaggggacacatttacggtgatatgcaccataaaagacgtgtctacatccgtggactccatgtggataaagttgaaccctcagcctcagagcaaagcccaggtaaagcgcaatagctggcatcagggcgacttcaattacgaacgccaggagacgctgactatcagctcagcaagagttaacgattccggagtgttcatgtgttatgccaataatacttttggatcagcaaatgtcacaacaaccttgaaagtagtagaaaagggattcatcaacatcttccctgtgaagaacactacggtatttgtaactgatggggaaaatgtagacttggttgttgagttcgaggcctaccctaaacctgaacaccagcagtggatctacatgaacaggacgcctactaacagaggggaggattatgtcaaatccgacaaccaaagcaacatcagatatgtgaacgaacttcgcctgaccagattgaaaggcacagaaggaggcacttacacctttctggtgtccaactctgatgtcagtgcttccgtgacatttgatgtttatgtgaacacaaaaccagaaatcctgacatatgacaggctcatgaatggcaggctccagtgtgtggcggcgggattcccggagcccacaatagattggtatttttgtacaggggcagagcaaaggtgtaccgttcctgtcccgccagtagacgtacagatccagaatgcgtctgtgtcaccatttggaaaactggtggttcagagttccatagactccagcgtcttccggcacaacggcacggtggagtgtaaggcctccaacgctgtgggcaagagctctgccttctttaactttgcatttaaaggtaacagcaaagagcaaatccagccccacaccctgttcacgccgctgctcattggcttcgtggtcacagccggcttgatggggatcattgtgatggttcttgcctacaaatatttgcagaaacccatgtatgaagtacaatggaaggttgtcgaggagataaatgggaacaattatgtttacatagacccaacgcagcttccttatgaccacaaatgggagtttcccagaaacaggctgagttttggaaagaccttgggagctggtgcctttgggaaggtagttgaggccactgcctatggcttaattaagtcggatgccgccatgacggttgccgtgaagatgctcaaaccaagtgcccatttaacggaaagggaggccctaatgtcagaactgaaggtcctgagctacctgggtaatcacatgaatatcgtcaacctccttggagcgtgtaccgtgggagggcccaccctggtcattacagaatactgttgctatggtgatcttttgaatttcttgagaagaaagcgtgactcgtttattttctcaaagcaagaagaacaggcagacgccgcactttataagaaccttctgcattcaaaggagtcttcctgtgacagctcaaacgagtacatggacatgaagcctggcgtttcctacgtcgtaccaaccaagacagacaaaaggagatccgcaagaatagactcgtatatagaaagagacgtgactcccgccatcatggaagatgacgagctggctctggacctggaagatttgctgagcttttcctaccaggtggccaagggcatggcgttcctcgcctccaagaactgtattcacagagatttggcagccaggaatatcctcctcactcacgggcggatcacaaagatttgcgatttcggcctagccagagacatcaggaatgattcgaattacgtggtaaaaggaaatgcacggctgcccgtgaagtggatggcaccggagagcattttcaactgcgtgtacacatttgaaagtgacgtctggtcctatgggattttcctctgggagctattctctctaggaagcagcccctacccagggatgccggtcgattccaagttttacaagatgatcaaggaaggtttccgaatgctcagccctgagcacgcgcctgccgcaatgtatgaagttatgaagacttgctgggatgctgatcccctgaaaaggccaacattcaagcaggttgttcagctcattgagaagcagatctcagacagcagcaaacatatttactccaacttagcaaactgtaaccccaacccagagaaccccgtggtggtggaccattctgtgagggtcaattccgtcggcagcagcacctcttccacacagcctctcctcgtgcatgaggacgcctgagtagaaacggagcccgatgggcattgctgtggtctccaaccccattctcctggcttctatgatggttattttgttttcctttgacttgcatcctactccagggtagcgggatccccgccccacccccaaccccactgtgattctgccttttatgagcacactttagtggctgatggcctttccttttcgccatcagccaccatcccaccaagaaggtccgaacggtatgtatatattttcccattagcaaagtagcccctactgtaaacggaaggcctcatgctttagaggaggaagggtagggtgcaacggggatgcctggagttcttcacagtgctcctccgagtgtgtttgaaaagtatggccagtagttcatttgaagagtttagaagtagtcccgttttggcccagagagccttccataatgacgggcagatgtatgtagcaagactagaaaggaaacccaagccctgtgtgtggaaagtagaccattattagaacagaggacacatgagaacatctaggcctaagaagtctggtcatgctgagaacgagacctaggctgagacggcgcaagccctggaagcgtggacatagatgctctgttctggggctgcgcgggcttttgcgcaagcttttgtacaactgacctggtttttaaatagtctgctgttggggagtagaattggagacaaggcctcctccctagccagcgtttgtatatactcactgtacgttgtatgcgttcatactttggagcgggggatccccccacaagctttagtttctgtgtacaaccctgggattaggtctgctgtgtgtaagaatagattta

SEQ ID NO: 6 - protein, 978 aa, Rattus norvegicus
MRGARGAWDLLCVLLVLLRGQTGTSQPSASPGEPSPPSIQPAQSELIVEAGDTIRLTCTDPAFVKWTFEILDVRIENKQSEWIREKAEATHTGKYTCVSGSGLRSSIYVFVRDPAVLFLVGLPLFGKEDNDALVRCPLTDPQVSNYSLIECDGKSLPTDLKFVPNPKAGITIKNVKRAYHRLCIRCAAQREGKWMRSDKFTLKVRAAIKAIPVVSVPETSHLLKEGDTFTVICTIKDVSTSVDSMWIKLNPQPQSKAQVKRNSWHQGDFNYERQETLTISSARVNDSGVFMCYANNTFGSANVTTTLKVVEKGFINIFPVKNTTVFVTDGENVDLVVEFEAYPKPEHQQWIYMNRTPTNRGEDYVKSDNQSNIRYVNELRLTRLKGTEGGTYTFLVSNSDVSASVTFDVYVNTKPEILTYDRLMNGRLQCVAAGFPEPTIDWYFCTGAEQRCTVPVPPVDVQIQNASVSPFGKLVVQSSIDSSVFRHNGTVECKASNAVGKSSAFFNFAFKGNSKEQIQPHTLFTPLLIGFVVTAGLMGIIVMVLAYKYLQKPMYEVQWKVVEEINGNNYVYIDPTQLPYDHKWEFPRNRLSFGKTLGAGAFGKVVEATAYGLIKSDAAMTVAVKMLKPSAHLTEREALMSELKVLSYLGNHMNIVNLLGACTVGGPTLVITEYCCYGDLLNFLRRKRDSFIFSKQEEQADAALYKNLLHSKESSCDSSNEYMDMKPGVSYVVPTKTDKRRSARIDSYIERDVTPAIMEDDELALDLEDLLSFSYQVAKGMAFLASKNCIHRDLAARNILLTHGRITKICDFGLARDIRNDSNYVVKGNARLPVKWMAPESIFNCVYTFESDVWSYGIFLWELFSLGSSPYPGMPVDSKFYKMIKEGFRMLSPEHAPAAMYEVMKTCWDADPLKRPTFKQVVQLIEKQISDSSKHIYSNLANCNPNPENPVVVDHSVRVNSVGSSTSSTQPLLVHEDA